SEQUENCE LISTING (10 sequences)

SEQ ID NO: 1 - DNA, 6765 bp, Homo sapiens
gaccgcggggtgggcggccccgagagcgtcccgcagtggctggagccctgggcgctgcaaagcgtgtcccgccgggtccccgagcgtcccgcgccctcgccccgccatgctcctgctgctggggctgtgcctggggctgtccctgtgtgtggggtcgcaggaagaggcgcagagctggggccactcttcggagcaggatggactcagggtcccgaggcaagtcagactgttgcagaggctgaaaaccaaacctttgatgacagaattctcagtgaagtctaccatcatttcccgttatgccttcactacggtttcctgcagaatgctgaacagagcttctgaagaccaggacattgagttccagatgcagattccagctgcagctttcatcaccaacttcactatgcttattggagacaaggtgtatcagggcgaaattacagagagagaaaagaagagtggtgatagggtaaaagagaaaaggaataaaaccacagaagaaaatggagagaaggggactgaaatattcagagcttctgcagtgattcccagcaaggacaaagccgcctttttcctgagttatgaggagcttctgcagaggcgcctgggcaagtacgagcacagcatcagcgtgcggccccagcagctgtccgggaggctgagcgtggacgtgaatatcctggagagcgcgggcatcgcatccctggaggtgctgccgcttcacaacagcaggcagaggggcagtgggcgcggggaagatgattctgggcctcccccatctactgtcattaaccaaaatgaaacatttgccaacataatttttaaacctactgtagtacaacaagccaggattgcccagaatggaattttgggagactttatcattagatatgacgtcaatagagaacagagcattggggacatccaggttctaaatggctattttgtgcactactttgctcctaaagaccttcctcctttacccaagaatgtggtattcgtgcttgacagcagtgcttctatggtgggaaccaaactccggcagaccaaggatgccctcttcacaattctccatgacctccgaccccaggaccgtttcagtatcattggattttccaaccggatcaaagtatggaaggaccacttgatatcagtcactccagacagcatcagggatgggaaagtgtacattcaccatatgtcacccactggaggcacagacatcaacggggccctgcagagggccatcaggctcctcaacaagtacgtggcccacagtggcattggagaccggagcgtgtccctcatcgtcttcctgacggatgggaagcccacggtcggggagacgcacaccctcaagatcctcaacaacacccgagaggccgcccgaggccaagtctgcatcttcaccattggcatcggcaacgacgtggacttcaggctgctggagaaactgtcgctggagaactgtggcctcacacggcgcgtgcacgaggaggaggacgcaggctcgcagctcatcgggttctacgatgaaatcaggaccccgctcctctctgacatccgcatcgattatccccccagctcagtggtgcaggccaccaagaccctgttccccaactacttcaacggctcggagatcatcattgcggggaagctggtggacaggaagctggatcacctgcacgtggaggtcaccgccagcaacagtaagaaattcatcatcctgaagacagatgtgcctgtgcggcctcagaaggcagggaaagatgtcacaggaagccccaggcctggaggcgatggagagggggacaccaaccacatcgagcgtctctggagctacctcaccacaaaggagctgctgagctcctggctgcaaagtgacgatgaaccggagaaggagcggctgcggcagcgggcccaggccctggctgtgagctaccgcttcctcactcccttcacctccatgaagctgagggggccggtcccacgcatggatggcctggaggaggcccacggcatgtcggctgccatgggacccgaaccggtggtgcagagcgtgcgaggagctggcacgcagccaggacctttgctcaagaagccataccagccaagaattaaaatctctaaaacatcagtggatggtgatccccactttgttgtggatttccccctgagcagactcaccgtgtgcttcaacattgatgggcagcccggggacatcctcaggctggtctctgatcacagggactctggtgtcacagtgaacggagagttaattggggcacccgcccctccaaatggccacaagaaacagcgcacttacttgcgcactatcaccatcctcatcaacaagccagagagatcttatctcgagatcacaccgagcagagtcatcttggatggtggggacagactggtgctcccctgcaaccagagtgtggtggtggggagctgggggctggaggtgtccgtgtctgccaacgccaatgtcaccgtcaccatccagggctccatagcctttgtcatcctcatccacctctacaaaaagccggcgcccttccagcgacaccacctgggtttctacattgccaacagcgagggcctttccagcaactgccacggactgctgggtcagttcctgaatcaggatgccagactcacagaagaccctgcagggcccagccagaacctcactcaccctctgctccttcaggtgggagaggggcctgaggccgtcctaacagtgaaaggccaccaagtcccagtggtctggaagcaaaggaagatttacaacggggaagagcagatagactgctggtttgccaggaacaatgccgccaaactgattgacggggagtacaaggattacctggcatcccatccatttgacacagggatgacacttggccggggaatgtccagggagctctgaagctggcagccttaaagatgcaagtgcatgaaggacagtgatgtggggaggccgtggggcagctcttttcatggcttgtacacgcctcagctcctggcaattagctggactccatgacccacccctggtgcagcatagatccgacgtctgtctgggcgaagggtaggggtgggtaggggcgggaagcctgagtgcaaatgtcatttccctctactgcctcttcctgcctctccccaccctgcccacatccacagaggggagagaagggtcatagctaaatgcaacaaagtctgtatcttgtcccaacctgcttttctgttctgttagcatatcataaagtaagcctttctggtgaaggaaggttgctatgaaactttttttcttggtggaaatggacaagtttaggcactctgctttttgccttacactaatgcttagaaagctgtcttttcagtggtgttgcagcccccagatgtgtggccaacctctgctgcaaaggaatctcttgctgagtccaggccaccaatcaggcaaatagcccatacatttgatcgttgtaaaccatgaagtcttttcttgcaagacgtttttcttctgctgtggtatcttgcccttaaaaattagttttcattaaaaagaaatttgattgaaaatagaaacatctacagctcagatactgatctctttctaatgggctttgtaaaccacttgactttgctaatggctattctccaatttgttccaataagctaagggcttttcatacacaggacaactccatgtggttgattttactggcatgtcttggtgacgaagagttggccaagtgaatgatgcatcttttacttggagctctccagcctttgtcatagttcccagaccttttttccttgtggtcatccaggaggtgatcaattacatgtgcacagtgctaggacgggaggcagagccggctcagagcaggtccaccagcccacctatagcaggaagcgtgtcggtgcttaggaaatctaggacttccagcttccctcatgtcagatagaaaaaaagtcaggaaaccagctcgtggacaggggctttggttccaaaacttggacaaacctgatgccttaaggaaaaggttaagatgagaggggacttccagagtcaatgaacgtcacccttcagcggacggataccgggagcgcttgggcgaaacagcagagaaagaaagactgtggtcggggaagtggaatgcttcgatgcaggccagagttcaaggctctggggctggcctcacaagaggcttcttccacggggcttgacaccagagcagcccccgcgtgaaatgagaaggcatgcttagcactgggaggaaaagctgagttaagaaaattctttatcatgtttgccttccctggggaaacattcaggcagtaaaatgacatccacgtgttcctgtcggacgcagcggcttcgggaacaagttagcgctgggggactatctgggctcgtctctctctgcttcgtgaacccaccacaccagggcctgtctgctgtgctatttatttgccttccgtcttcccaacctcagctgcctgtattcatgccagaggtgcaaaggctgccagccagtgctgctgacagatgctttcagactgtagagctcagagaaagggagcggcagctagaggtagccagggagctggccaggctccccggggccttcctattatgtaaatcccagctggacagacagccctggagccaggtggccctgtcccctgtggagcacttggagcagtgatctgttgttccaggtgtaagtctcgacactgcagaaatgaggacgttattaggaaagaccagttcctactgcctgtgttgctactttaagagctgagaaaaacattccaatgagctatccatagctaacttgcttgctcagaaaaacataagtaacaaagttgagctttcatttttaaaaattatattttactttagaaaaatatcaccttttttcttcctcttctttacgtgtagatcttttttttttttttcaaatggggtcagtacctgaaagcccgaaaccccataccaagcttatagttaggacctttgcatttgataaaaaaaaaatatagtattttctgttgtccagaaaatcttcccagatagggaaactgaggcagagagtgccgtggagtggtttttgccaagtcatacaacatacttaatgcagggctacactgaacctccagaatcattttgtttgctttgaccctgtttcaccatctgggcaatcatttattttccttgagcatttataaaccagggcgagggttgttccaaaatgagtaattgaatgagatgaagaaatatgtcataattagaggagataaatctccttgttatcacaatagttaatgtgtcatgatcactgactaactgggaccttgaacaagtcagcatctctgcctttcaaaatgaaggaacattgttctctatcctcccctgacccccgacggccccatgctaggtgcaccatcttcctccatttgactttttaatgctttacaacttcaggctaaagggaggggatgttgggaggagaaaccatcacaagattccctcggtgccacagctgtgctgaggaggccggggactgggaatcggagtgggcacctctgtgtttctctagggagtaacattcaacatttaattgaatcgtgtgtcctgataggtggattaggaacaggattcattcctttaactttaacttttggctaaacgacatgccttggattgagtgtatatgtcttccccaaattcatatgttgaagcttaaatcctgaatgtgatggttcttggaattgggacctttggaaggttattgggtcttgagggcagagccctcacgaatgggattaaggcccttataagaagagacatggggccaggtgcagtggctcacacctgtaatcccaacattttgggaggccgagaagggtggatcatgaggtcaggagatggagaccatcctggccaacatggtgaaacctcgtctctactaaaaatacaaaaattagctgggcgtggtggtgcgtgcctgtagttccagctacttgggaggctgaggcaggagaatcgcttgaaccaggcagtcggaacttgtagtgagccaagatcacgccactgcactccagctggagtgacagagccaaactccgtcaaaaaaaaacaaaacaaaacaaaaaaaagacatgggagagatggtgtttttcctctccaccacctggggatacgatgagaagatggccacctgcaagccaggaagacggccctcaccagacaccatgtctgctggcaccttgatcttggacctcccagcctccagaactgtgagaaataaatgtgttttgtttaagctactcaagtctgtggtattcttgttaaggcagcccaagctgactaagacaccaaatcattcaaaacgttaattctttttctgttctttaagcttcccagaatgtacacaaacaatgctgttattaaaatttaaaggactcttggaactgtaggacatgatccagctaaaataaataacaaataacacagctaagcaaatacttataaatgtagctttaaatatttattttagttttaaataattagtggggtacgagacagttcacattcaagagacattttacttagacaaaaaatttataagagaacgatgcataaaaacaaacaacatattggtctgtttatttttctaagggaaaatggaattggagctgttgagaataaaacttgaagaatctaactgcaaaaaaaaaaaaaaaaaa

SEQ ID NO: 2 - DNA, 3132 bp, Homo sapiens
gaccgcggggtgggcggccccgagagcgtcccgcagtggctggagccctgggcgctgcaaagcgtgtcccgccgggtccccgagcgtcccgcgccctcgccccgccatgctcctgctgctggggctgtgcctggggctgtccctgtgtgtggggtcgcaggaagaggcgcagagctggggccactcttcggagcaggatggactcagggtcccgaggcaagtcagactgttgcagaggctgaaaaccaaacctttgatgacagaattctcagtgaagtctaccatcatttcccgttatgccttcactacggtttcctgcagaatgctgaacagagcttctgaagaccaggacattgagttccagatgcagattccagctgcagctttcatcaccaacttcactatgcttattggagacaaggtgtatcagggcgaaattacagagagagaaaagaagagtggtgatagggtaaaagagaaaaggaataaaaccacagaagaaaatggagagaaggggactgaaatattcagagcttctgcagtgattcccagcaaggacaaagccgcctttttcctgagttatgaggagcttctgcagaggcgcctgggcaagtacgagcacagcatcagcgtgcggccccagcagctgtccgggaggctgagcgtggacgtgaatatcctggagagcgcgggcatcgcatccctggaggtgctgccgcttcacaacagcaggcagaggggcagtgggcgcggggaagatgattctgggcctcccccatctactgtcattaaccaaaatgaaacatttgccaacataatttttaaacctactgtagtacaacaagccaggattgcccagaatggaattttgggagactttatcattagatatgacgtcaatagagaacagagcattggggacatccaggttctaaatggctattttgtgcactactttgctcctaaagaccttcctcctttacccaagaatgtggtattcgtgcttgacagcagtgcttctatggtgggaaccaaactccggcagaccaaggatgccctcttcacaattctccatgacctccgaccccaggaccgtttcagtatcattggattttccaaccggatcaaagtatggaaggaccacttgatatcagtcactccagacagcatcagggatgggaaagtgtacattcaccatatgtcacccactggaggcacagacatcaacggggccctgcagagggccatcaggctcctcaacaagtacgtggcccacagtggcattggagaccggagcgtgtccctcatcgtcttcctgacggatgggaagcccacggtcggggagacgcacaccctcaagatcctcaacaacacccgagaggccgcccgaggccaagtctgcatcttcaccattggcatcggcaacgacgtggacttcaggctgctggagaaactgtcgctggagaactgtggcctcacacggcgcgtgcacgaggaggaggacgcaggctcgcagctcatcgggttctacgatgaaatcaggaccccgctcctctctgacatccgcatcgattatccccccagctcagtggtgcaggccaccaagaccctgttccccaactacttcaacggctcggagatcatcattgcggggaagctggtggacaggaagctggatcacctgcacgtggaggtcaccgccagcaacagtaagaaattcatcatcctgaagacagatgtgcctgtgcggcctcagaaggcagggaaagatgtcacaggaagccccaggcctggaggcgatggagagggggacaccaaccacatcgagcgtctctggagctacctcaccacaaaggagctgctgagctcctggctgcaaagtgacgatgaaccggagaaggagcggctgcggcagcgggcccaggccctggctgtgagctaccgcttcctcactcccttcacctccatgaagctgagggggccggtcccacgcatggatggcctggaggaggcccacggcatgtcggctgccatgggacccgaaccggtggtgcagagcgtgcgaggagctggcacgcagccaggacctttgctcaagaagccataccagccaagaattaaaatctctaaaacatcagttcgtggcttcagaagggaagaacagattttttcaaatgtcatgaaatttccttttgccagcatcctggaactataataaaaattcagaggaaagaacaacctatgactgggttttgttctgctgtgtccagatcatcctctggaagtcttgattgttgcctttctgctgctgtgctgtacgtgcaagttcctaaagccagggacatcccggagatgagctgtggatatagagtccgcacagcctgcgggagacagaatgtaattccttccaacacacacacacaggctttatggaatgccagaaaagcttcctcttggatgctgcctggagtaatgactttttgaccctcccctcttttttggagcaagcactatctttccttgtaaagaaatttggacaatttgagtagaaatggtgtggtatgtgcacatagaaactctcagaaaactgtcagatgagctagacagttgtcctctatctccatgggtcccgcatcccaaggatcgaaccaacaacagatcaaaaatacgtgaaaaaggaattgcatctgtattgaatgtgtacggactttttttcttgtctttgttccttaaacaatgtagtgtaacaactatttacatcgcatctcctttgcattcattattataagtaaactagagatgatttaaagtgtatagaaaggctgcgtgcagtggctaatgtctgtaatcccagcactttgagaggccgaggcaggtggatcacctgaggtcaggagttcgagaccagcctggccaacgtggtgaaaccccatctctactaaaaatacaataattagctgggcatggtggtgcacgcctgtaatcccagctattcgggaggctgaggcaggagaatcgcttgaacccaggaggcagagattgcagtgaaccgagattgcaccattgcactccagcctgggtgacagagtgaacctccgtctcaaaaacaaaataaataaataagaataaatt

SEQ ID NO: 3 - DNA, 3132 bp, Homo sapiens; identical to SEQ ID NO: 2

SEQ ID NO: 4 - protein, 942 aa, Homo sapiens
MLLLLGLCLGLSLCVGSQEEAQSWGHSSEQDGLRVPRQVRLLQRLKTKPLMTEFSVKSTIISRYAFTTVSCRMLNRASEDQDIEFQMQIPAAAFITNFTMLIGDKVYQGEITEREKKSGDRVKEKRNKTTEENGEKGTEIFRASAVIPSKDKAAFFLSYEELLQRRLGKYEHSISVRPQQLSGRLSVDVNILESAGIASLEVLPLHNSRQRGSGRGEDDSGPPPSTVINQNETFANIIFKPTVVQQARIAQNGILGDFIIRYDVNREQSIGDIQVLNGYFVHYFAPKDLPPLPKNVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEIRTPLLSDIRIDYPPSSVVQATKTLFPNYFNGSEIIIAGKLVDRKLDHLHVEVTASNSKKFIILKTDVPVRPQKAGKDVTGSPRPGGDGEGDTNHIERLWSYLTTKELLSSWLQSDDEPEKERLRQRAQALAVSYRFLTPFTSMKLRGPVPRMDGLEEAHGMSAAMGPEPVVQSVRGAGTQPGPLLKKPYQPRIKISKTSVDGDPHFVVDFPLSRLTVCFNIDGQPGDILRLVSDHRDSGVTVNGELIGAPAPPNGHKKQRTYLRTITILINKPERSYLEITPSRVILDGGDRLVLPCNQSVVVGSWGLEVSVSANANVTVTIQGSIAFVILIHLYKKPAPFQRHHLGFYIANSEGLSSNCHGLLGQFLNQDARLTEDPAGPSQNLTHPLLLQVGEGPEAVLTVKGHQVPVVWKQRKIYNGEEQIDCWFARNNAAKLIDGEYKDYLASHPFDTGMTLGRGMSREL

SEQ ID NO: 5 - protein, 728 aa, Homo sapiens
MRNYDSGPPPSTVINQNETFANIIFKPTVVQQARIAQNGILGDFIIRYDVNREQSIGDIQVLNGYFVHYFAPKDLPPLPKNVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEIRTPLLSDIRIDYPPSSVVQATKTLFPNYFNGSEIIIAGKLVDRKLDHLHVEVTASNSKKFIILKTDVPVRPQKAGKDVTGSPRPGGDGEGDTNHIERLWSYLTTKELLSSWLQSDDEPEKERLRQRAQALAVSYRFLTPFTSMKLRGPVPRMDGLEEAHGMSAAMGPEPVVQSVRGAGTQPGPLLKKPYQPRIKISKTSVDGDPHFVVDFPLSRLTVCFNIDGQPGDILRLVSDHRDSGVTVNGELIGAPAPPNGHKKQRTYLRTITILINKPERSYLEITPSRVILDGGDRLVLPCNQSVVVGSWGLEVSVSANANVTVTIQGSIAFVILIHLYKKPAPFQRHHLGFYIANSEGLSSNCHGLLGQFLNQDARLTEDPAGPSQNLTHPLLLQVGEGPEAVLTVKGHQVPVVWKQRKIYNGEEQIDCWFARNNAAKLIDGEYKDYLASHPFDTGMTLGRGMSREL

SEQ ID NO: 6 - protein, 702 aa, Homo sapiens
MLLLLGLCLGLSLCVGSQEEAQSWGHSSEQDGLRVPRQVRLLQRLKTKPLMTEFSVKSTIISRYAFTTVSCRMLNRASEDQDIEFQMQIPAAAFITNFTMLIGDKVYQGEITEREKKSGDRVKEKRNKTTEENGEKGTEIFRASAVIPSKDKAAFFLSYEELLQRRLGKYEHSISVRPQQLSGRLSVDVNILESAGIASLEVLPLHNSRQRGSGRGEDDSGPPPSTVINQNETFANIIFKPTVVQQARIAQNGILGDFIIRYDVNREQSIGDIQVLNGYFVHYFAPKDLPPLPKNVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEIRTPLLSDIRIDYPPSSVVQATKTLFPNYFNGSEIIIAGKLVDRKLDHLHVEVTASNSKKFIILKTDVPVRPQKAGKDVTGSPRPGGDGEGDTNHIERLWSYLTTKELLSSWLQSDDEPEKERLRQRAQALAVSYRFLTPFTSMKLRGPVPRMDGLEEAHGMSAAMGPEPVVQSVRGAGTQPGPLLKKPYQPRIKISKTSVRGFRREEQIFSNVMKFPFASILEL

SEQ ID NO: 7 - protein, 460 aa, Homo sapiens
DSGPPPSTVINQNETFANIIFKPTVVQQARIAQNGILGDFIIRYDVNREQSIGDIQVLNGYFVHYFAPKDLPPLPKNVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEIRTPLLSDIRIDYPPSSVVQATKTLFPNYFNGSEIIIAGKLVDRKLDHLHVEVTASNSKKFIILKTDVPVRPQKAGKDVTGSPRPGGDGEGDTNHIERLWSYLTTKELLSSWLQSDDEPEKERLRQRAQALAVSYRFLTPFTSMKLRGPVPRMDGLEEAHGMSAAMGPEPVVQSVRGAGTQPGPLLKKPYQPRIKISKTSV

SEQ ID NO: 8 - protein, 264 aa, Homo sapiens
DGDPHFVVDFPLSRLTVCFNIDGQPGDILRLVSDHRDSGVTVNGELIGAPAPPNGHKKQRTYLRTITILINKPERSYLEITPSRVILDGGDRLVLPCNQSVVVGSWGLEVSVSANANVTVTIQGSIAFVILIHLYKKPAPFQRHHLGFYIANSEGLSSNCHGLLGQFLNQDARLTEDPAGPSQNLTHPLLLQVGEGPEAVLTVKGHQVPVVWKQRKIYNGEEQIDCWFARNNAAKLIDGEYKDYLASHPFDTGMTLGRGMSREL

SEQ ID NO: 9 - protein, 218 aa, Homo sapiens
MLLLLGLCLGLSLCVGSQEEAQSWGHSSEQDGLRVPRQVRLLQRLKTKPLMTEFSVKSTIISRYAFTTVSCRMLNRASEDQDIEFQMQIPAAAFITNFTMLIGDKVYQGEITEREKKSGDRVKEKRNKTTEENGEKGTEIFRASAVIPSKDKAAFFLSYEELLQRRLGKYEHSISVRPQQLSGRLSVDVNILESAGIASLEVLPLHNSRQRGSGRGED

SEQ ID NO: 10 - protein, 16 aa, Mus musculus
YDEIRTPLLSDIRIDY

(19) United States
(12) Patent Application Publication
(10) Pub. No.: US 2015/0038778 A1
(43) Pub. Date: Feb. 5, 2015

(54) ITIH5 AS A DIAGNOSTIC MARKER OF UTERINE DEVELOPMENT AND FUNCTIONAL DEFECTS

(71) Applicant: Cewntree Hospitalier Universitaire Pontchaillou, Rennes (FR)

(72) Inventors: Daniel Guerrier, Mouaze (FR); Karine Morel, Rennes (FR)

(21) Appl. No.: 14/384,336

(22) PCT Filed: Mar. 14, 2013
(86) PCT No.: PCT/EP2013/005527

§ 371 Date: Sep. 10, 2014

Related U.S. Application Data
(60) Provisional application No. 61/610,590, filed on Mar. 14, 2012.

(51) Int. Cl. G01N 33/68; A61B 17/435; A61D 19/04; C12Q 1/68

(52) U.S. Cl. CPC G01N 33/689; C12Q 1/6883; A61B 17/435; A61D 19/04; C12Q 2600/118; C12Q 2600/158; G01N 2800/367; G01N 2800/364; G01N 2333/4722
USPC 600/34; 436/501; 435/7.92; 435/7.1; 435/6.11; 435/6.12

(57) ABSTRACT

The invention relates to the use of ITIH5 as a biomarker of endometrium receptivity to embryo implantation. Also provided are methods and kits for using this biomarker for detecting the receptivity state of the endometrium of a mammal to embryo implantation, for diagnosing infertility in a female mammal, for the early diagnosis of pregnancy, for detecting the window of embryo implantation in the endometrium of a female mammal, for in vitrofertilization of a female mammal, and for diagnosing endometriosis.

### RELATED PATENT APPLICATION

The present application claims priority to U.S. Provisional Patent Application No. 61/610,590 filed on Mar. 14, 2012. The Provisional patent application is incorporated herein by reference in its entirety.

### BACKGROUND OF THE INVENTION

The relative inefficiency of human reproduction is reflected in a high prevalence of pre-implantation embryo losses, pre-clinical pregnancy losses and clinical miscarriages. Only about 30% of all conceptions survive to birth and 55% are lost in the early stages of pregnancy. This high rate of early loss is considered to represent a natural strategy for dealing with the high prevalence of chromosomal abnormalities in human embryos. The other 15% of conceptions end in recognizable miscarriage. Inadequate uterine receptivity and subsequent embryo implantation failure, rather than fertilization failure, has been implicated as the crucial event which differentiates fertile and non-fertile ovulatory cycles.

Embryo implantation into the endometrium (i.e., the mucosa coating the inside of the uterine cavity) requires a receptive endometrium capable of responding to the signals of the blastocyst, which is the stage of development of the embryo when it implants. Human endometrium is a tissue cyclically regulated by hormones, the hormones preparing it to reach said receptivity state are estradiol, which induces cell proliferation, and progesterone, which is involved in differentiation, causing a large number of changes in the gene expression profile of the endometrium, which reaches a receptive phenotype for a short period referred to as “window of implantation”. Although there is no consensus as to the implantation period in humans, clinical studies suggest that during an idealized 28-day human menstrual cycle, the endometrium is only receptive for a short two-day period during luteal days 20-24. If the endometrium is not receptive to implantation, implantation of a fertilized egg may not occur or may occur in an abnormal manner. If the endometrium is receptive, implantation is optimized. Accordingly, it would be useful to have a diagnostic tool for determining endometrial receptivity.

The importance of endometrial adequacy and receptivity has become even more apparent with the evolution of assisted reproduction technologies. In vitro fertilization and embryo transfer procedures produce fertilization rates of 70% to 90%. However, pregnancy rates after embryo transfer remain disappointingly low ranging from 15% to 25%. In addition, there appears to be a high incidence of early pregnancy loss after in vitro fertilization with a biochemical pregnancy rate of 18% and a spontaneous abortion rate of 27%. Inaccurate identification of the window of implantation is a major cause for the low success rate in artificial reproductive technologies. Furthermore, ovarian hyperstimulation protocols used for these procedures have been associated with several factors that may contribute to lower implantation rates by causing adverse changes in endometrium receptivity. Unfortunately, no non-invasive methods or markers have yet been developed to detect the effects of these protocols on subsequent uterine receptivity.

Improved knowledge of factors influencing human embryo implantation and an accurate marker of endometrium receptivity would help reduce embryo loss by reducing the number of embryos needed for transfer and reducing the potential of multiple gestation. Indeed, an accurate marker would allow monitoring of the state of uterine receptivity prior to embryo transfer. Embryo transfer procedures could be timed to coincide with a receptive endometrium or could be delayed, cryopreserving embryos, until a more appropriate state of receptivity is attained. This may be especially relevant in patients where ovarian hyperstimulation and ovulation induction have altered the natural course of endometrial development. A marker of uterine receptivity may also be diagnostic in women who repeatedly fail to become pregnant and who have no other apparent etiology for their infertility.

Therefore, there is a need in the art for methods for determining endometrial receptivity to embryo implantation. There is also a need for markers to identify a woman's relative risk of experiencing a spontaneous abortion early in pregnancy. The present invention addresses these needs.

### SUMMARY OF THE INVENTION

The present invention generally relates to systems and improved strategies for accurately determining the state of the endometrium, which allow the endometrium to be monitored for embryo receptivity, embryo implantation and infertility, and for diagnosing endometriosis. More specifically, the invention is based upon the discovery that ITIH5, a member of the inter-α-trypsin inhibitor (ITI) protein family, is expressed in a temporal pattern in the endometrium of female subjects of reproductive age. As described in more detail in the Examples provided below, ITIH5 was found to be highly expressed in the developing uterus of mouse embryo. In adult female mice, ITIH5 expression was found to vary during the estrous cycle and during gestation. A peak of expression of ITIH5 was observed during the estrus phase of the estrous cycle. During gestation, a first peak of expression of ITIH5 was observed at the implantation stage. Then a second peak was observed mid-gestation, corresponding to the myometrial growth.

The temporal pattern of ITIH5 expression in the endometrium during the menstrual/estrous cycle and during gestation makes ITIH5 useful as a biomarker allowing the endometrium to be monitored for embryo receptivity and embryo implantation.

In one aspect, the present invention provides a method for detecting endometrial receptivity to embryo implantation in a female mammal, the method comprising steps of: (a) measuring the level of expression of ITIH5 in a biological sample obtained from the female mammal; and (b) correlating the level of expression of ITIH5 in the biological sample with endometrial receptivity to embryo implantation in said female subject. In some embodiments, the method comprises detecting the level of expression of ITIH5 in biological samples obtained from a plurality of stages of the menstrual or estrous cycle of the female mammal.

In this method, as well as in the other methods of the present invention, the female mammal may be any female placental mammal, including humans. In certain embodiments, the female placental mammal is a non-human female mammal selection from domesticated placental mammals, such as, for example, mammalian farm animals, mammalian sport animals or mammalian pets.

In this method, as well as in the other methods of the present invention, the biological sample may be any biological sample allowing ITIH5 to be assayed. In certain preferred embodiments, the biological sample is a biological fluid selected from the group consisting of blood (e.g., whole blood, serum or plasma), urine, uterine/cervical secretions, and vaginal secretions. Preferably, the biomarker ITIH5 is detected in blood, serum or urine.

In certain embodiments of this method, as well as in the other methods of the present invention, the step of measuring the level of expression of ITIH5 in a biological sample obtained from the female mammal comprises measuring the DNA or RNA expression of the ITIH5 gene.

In other embodiments of this method, as well as in the other methods of the present invention, the step of measuring the level of expression of ITIH5 in a biological sample obtained from the female mammal comprises measuring the expression of a polypeptide encoded by the ITIH5 gene. Such measurement may be performed using an ELISA or immunoassay, for example using an antibody specific for ITIH5. In embodiments where endometrium receptivity is concerned, the antibody specifically binds to isoform 2 of the ITIH5 protein (i.e., to the adult isoform). In embodiments wherein endometriosis is concerned, the antibody specifically binds to isoform 3 of the ITIH5 protein (i.e., to the embryonic isoform).

In another aspect, the present invention provides a method for detecting the window of embryo implantation in a female mammal, the method comprising steps of: (a) providing or obtaining a biological sample from said female mammal; (b) measuring the expression level of ITIH5 in the biological sample obtained from the female mammal; (c) repeating steps (a) and (b) with biological samples obtained from a plurality of stages of the menstrual or estrous cycle of said female mammal; and (d) determining the period of time of the menstrual or estrous cycle when the endometrium of said female subject is available for embryo implantation, based on the levels of expression measured.

In another aspect, the present invention provides a method for diagnosing infertility in a female placental mammal, comprising steps of: (a) providing or obtaining a biological sample from said female mammal; (b) measuring the expression level of ITIH5 in the biological sample obtained from the female mammal; and (c) correlating the level of expression of ITIH5 in the biological sample with infertility in said female mammal. In certain embodiments, the method for diagnosing infertility comprises detecting the level of expression of ITIH5 in biological samples obtained from a plurality of stages of the menstrual or estrous cycle of the female mammal. In certain embodiments, the method for diagnosing infertility comprises determining the period of time of the menstrual or estrous cycle when the endometrium of said female subject is available for embryo implantation, based on the levels of expression measured.

When infertility is diagnosed using a method according to the invention, the method may further comprise a step of prescribing an appropriate treatment and/or of administering an appropriate treatment.

In another aspect, the present invention provides a method for monitoring the effects of a treatment protocol to induce ovarian hyperstimulation or ovulation induction on endometrium receptivity in a female mammal, comprising steps of: (a) providing or obtaining a biological sample from said female mammal; (b) measuring the expression level of ITIH5 in the biological sample obtained from the female mammal; and (c) detecting and/or identifying, in said female mammal, an alteration of the endometrium receptivity due to the treatment, based on the expression level measured. In certain embodiments, the method further comprises determining the period of time of the menstrual or estrous cycle when the endometrium of said female mammal is available for embryo implantation, based on the levels of expression measured.

The present invention also provides a method for assisted reproduction in a female placental mammal, comprising steps of: (a) measuring the expression level of ITIH5 in a biological sample obtained from the female mammal; (b) repeating step (a) with biological samples obtained from a plurality of stages of the menstrual or estrous cycle of said female mammal; (c) correlating the level of expression of ITIH5 in one or more samples of step (b) with endometrial maturation; and (d) transferring or introducing at least one embryo into the uterus of said female mammal when said endometrium is mature.

In certain embodiments, the embryo that is transferred develops from a zygote formed by the combination of an egg and sperm in vitro.

In certain embodiments, the method of assisted reproduction further comprises monitoring the transferred embryo for implantation.

In another aspect, the present invention provides a method for assessing the probability of success of embryo implantation in the endometrium of a female mammal following a naturally achieved conception or a conception resulting from assisted reproduction technology, comprising steps of: (a) measuring the expression level of ITIH5 in a biological sample obtained from the female mammal; and (b) correlating the level of expression of ITIH5 in said biological sample with the probability of success of embryo implantation in the endometrium of said female mammal. In certain embodiments, the method comprises measuring the level of expression of ITIH5 in biological samples obtained from a plurality of stages of the menstrual or estrous cycle of the female mammal.

In yet another aspect, the present invention provides a method for diagnosing pregnancy in a woman, comprising steps of: (a) detecting the level of expression of ITIH5 in a biological sample obtained from the subject; and (b) correlating the level of expression of ITIH5 in the biological sample with embryo implantation and diagnosing pregnancy. In this method, the biological sample is preferably blood, serum or urine.

In certain embodiments, the method for diagnosing pregnancy in a woman may further comprise a step of: detecting and/or measuring the expression level of one or more hormones selected from the group consisting of hormone folliculostimulante (FSH), luteinizing hormone (LH), progesterone, anti-Müllerian hormone (AMH), estradiol, beta-human chorionic gonadotropin (β-hCG) and any combination thereof.

The present invention also provides a method for the early diagnosis of endometriosis in a female mammal comprising steps of: (a) measuring the level of expression of ITIH5 in a sample of biological fluid obtained from the female mammal; and (b) correlating the level of expression of ITIH5 in the biological sample with a diagnosis of endometriosis.

In another aspect, the present invention relates to the use of ITIH5 as a biomarker of endometrial receptivity to embryo implantation in a female mammal or as a biomarker of the window of embryo implantation in a female mammal.

In yet another aspect, the present invention relates to the use of a reagent that specifically detects ITIH5 expression levels or of a kit comprising at least one reagent that specifically detects ITIH5 expression levels, for detecting endometrial receptivity to embryo implantation in a female mammal, or for detecting the window of embryo implantation in a female mammal, or for diagnosing infertility in a female mammal, or for monitoring the effects of a treatment protocol to induce ovarian hyperstimulation or ovulation induction on endometrium receptivity in a female mammal, or for assisted reproduction in a female mammal, or for assessing the probability of success of embryo implantation in the endometrium of a female mammal following a naturally achieved conception or a conception resulting from assisted reproduction technology, or for diagnosing pregnancy in a female mammal, or for the early diagnosis of endometriosis in a female mammal.

In certain embodiments, the reagent that specifically detects ITIH5 expression levels comprises an antibody that specifically binds ITIH5. The antibody may specifically bind the three isoforms of ITIH5, or may specifically bind either isoform 2 or isoform 3 of the ITIH5 protein.

The invention further provides a home pregnancy test kit comprising at least one test strip, wherein the at least one test strip is sensitive to the presence of ITIH5, in urine, and changes color, or otherwise indicates, when above the threshold sensitivity to ITIH5 is detected.

These and other objects, advantages and features of the present invention will become apparent to those of ordinary skill in the art having read the following detailed description of the preferred embodiments.

### BRIEF DESCRIPTION OF THE DRAWING

FIG. 1. Itih5 mRNA levels in the female genital tract and liver during development. RT-PCR analysis of Itih5 mRNA (A) in the female genital tract and (B) in fetal liver. Equal amounts of total RNA from each tissue, without the addition of reverse transcriptase (−), were used as negative controls. The position of the expected transcript (316 bp) is indicated by an arrow. (C) RT-qPCR analysis of Itih5 mRNA levels. The values for the expression of the gene was normalized to Hprt1, used as an internal control, and are plotted on the y-axis as multiples of the lowest value for this gene (that in the liver at E14.5). Error bars are derived from three independent experiments. E: embryonic day; ITIH: inter-α-trypsin inhibitor heavy chain; P0: day 0 post-partum.

FIG. 2. In situ hybridization analysis of Itih5 mRNA in the female genital tract of embryonic mice at E16.5 and E18.5 (ventral views). Itih5 expression was detected only in the cranial part of the Müllerian ducts (white arrow). a: adrenal gland; k: kidney; o: ovary; od: oviduct; u: uterine horns; ur: ureter; scale bars: 500 microns.

FIG. 3. RT-qPCR analysis of Itih5 mRNA in the adult uterus during pregnancy and the estrus cycle: comparison with mRNA levels in the placenta. The values for the expression of the gene were normalized to Hprt1, used as an internal control, and are plotted on the y-axis as multiples of the lowest value for this gene (that in the second day (D2) of the estrus cycle). Error bars were derived from three independent experiments. D: day; E: embryonic day; ITIH: inter-α-trypsin inhibitor heavy chain; P0: day 0 post partum.

FIG. 4. Western-blot analysis of Itih5 protein with the α-Itih5 antibody. (A) Characterization of the α-Itih5 antibody. Western-blot analysis of protein extracts from E. coli strain BL21 producing the pGEX-3×/Itih5 recombinant protein. Lane 1: Protein extract without IPTG induction; Lane 2: Protein extract with IPTG induction. The arrow indicates the molecular size of the specific signal for the GST-Itih5 protein (approximately 105 kDa). (B) Western-blot characterization of the Itih5 isoforms produced in the female genital tract and the liver at E18.5. Arrows indicate the molecular sizes of the specific bands found in the female genital tract (105 and 50 kDa) and in the liver (73 kDa). (C) Size characterization of the Itih5 isoform produced in the uterus of pregnant and non-pregnant adult mice. The arrow indicates the molecular size of the specific band found in the uterus during gestation and in non-pregnant animals (73 kDa). E: embryonic day; NP: non-pregnant.

FIG. 5. The graph shows the concentrations of ITIH5 measured in the serum of 157 women (see Example 2 for more details) and of 3 men. Diamonds represent the levels of ITIH5 measured for the samples obtained from women, and open circles represent the levels of ITIH5 measured for the samples obtained from men (each of the diamond and circles represents the concentration measured for one sample).

### DEFINITIONS

Throughout the specification, several terms are employed that are defined in the following paragraphs.

The term “infertility”, as used herein, refers to the biological inability of a subject to contribute to conception, as well as to the inability of a female subject to carry a pregnancy to full term.

The terms “assisted reproductive technology” and “assisted reproduction technology” are used herein interchangeably and refer to technology that assists in achieving pregnancy, including, but not limited to, in vitro fertilization (IVF), embryo transfer (e.g., transfer of embryos at any stage, including blastocysts), gamete intrafallopian transfer (GIFT), tubal embryo transfer (TET), intracytoplasmic sperm injection (ICSI) and intrauterine insemination (IUI).

As used herein, the term “subject” refers to a placental mammal including humans. In the context of the present, the subject is generally a female mammal Examples of placental mammals include, but are not limited to, humans, murines, simians, felines, canines, equines, bovines, and the like. In certain embodiments of the present invention, the placental mammal is selected from mammalian farm animals, mammalian sport animals, and mammalian pets. In certain important embodiments of the present invention, the placental mammal is a human being. In such embodiments, the subject is often referred to as an “individual” or a “patient”. The terms “subject”, “individual” and “patient” do not denote a particular age. The term “embryo” refers to mammalian embryos, including human embryos.

The terms “biomarker” and “marker” are used herein interchangeably. They refer to a substance that is a distinctive indicator of a biological process, biological event and/or physiopathological condition. The expression of the gene ITIH5 has been identified by the present inventors as indicative of endometrium state. As used herein, the term “indicative of endometrium state” refers to a biological process or event, which is diagnostic of endometrium state, in that the biological process or event is found significantly more often in subject with a given endometrium state or a given endometrium-associated disease or condition than in a subject with a different endometrium state or with a different endometrium-associated disease or condition (as determined using routine statistical methods).

As used herein, the term “endometrium” has its art understood meaning and refers to a glandular layer of variable thickness that lines the uterine inner wall (myometrium) of a female mammal. The endometrium is extremely sensitive to the hormones estrogen and progesterone and is composed of several functional layers. The basalis layer is nearest the myometrium and the functionalis is the layer closer to the surface. This tissue is made of epithelial cells, stromal (or mesenchymal) cells, and endometrium leukocytes.

In women of reproductive age, the endometrium undergoes cyclical developmental changes based on the ovarian cycle of hormone release. The proliferative stage of endometrial development for women is represented by cycle days 1-13 of an idealized 28 day menstrual cycle. A surge of gonadotropin luteinizing hormone (LH) occurs on day 14, with ovulation occurring on day 15 (LH+1). Secretory phases are: (1) early secretory for cycle days (LH+1 to LH+5); (2) mid-secretory for cycle days 20-24 (LH+6 to LH+10); and (3) late secretory for cycle days 25-28 (LH+11 to LH+14). The timing of embryo implantation and corresponding window of endometrial receptivity to embryo implantation is between cycle days 20-24 (LH+6 to LH+10).

As used herein, the terms “endometrial receptivity to embryo implantation” or “mature endometrium” refer to the state of the endometrium during the window of endometrial receptivity. In a woman, the “optimum timing window of embryo implantation” or “window of endometrial receptivity” or “window of implantation” refer to the time period between days 20 (LH+6) to 24 (LH+10) of an idealized 28 day human menstrual cycle. Similar cycles are known for other placental mammals and it is within the ordinary skill in the art to adopt methods described herein to such cycles. As known in the art, while women and non-human primates of reproductive age have menstrual cycles, female mammals of other species have estrous cycles.

The term “biological sample” is used herein in its broadest sense. A biological sample is generally obtained from the subject. A biological sample may be of any biological tissue or fluid with which the biomarker of the present invention may be assayed. Examples of biological samples suitable for use in the present invention include, but are not limited to, bodily fluids which may or may not contain cells, e.g., blood (e.g., whole blood, serum or plasma), urine, saliva, uterine/cervical secretions, and vaginal secretions, and biological tissues such as endometrium tissue, e.g., tissue or fine needle biopsy samples, and archival samples with known diagnosis, treatment and/or outcome history. Biological samples may also include sections of tissues such as frozen sections taken for histological purposes. The term “biological sample” also encompasses any material derived by processing a biological sample. Derived materials include, but are not limited to, cells (or their progeny) isolated from the sample, as well as proteins or nucleic acid molecules extracted from the sample. Processing of a biological sample may involve one or more of: filtration, distillation, extraction, concentration, inactivation of interfering components, addition of reagents, and the like.

As indicated above, the expression of the gene ITIH5 has been identified by the present inventors as indicative of endometrium state, in particular of endometrium receptivity. As used herein, the term “gene” refers to a polynucleotide that encodes a discrete macromolecular product, be it a RNA or a protein, and may include regulatory sequences preceding (5′ non-coding sequences) and following (3′ non-coding sequences) the coding sequence. As more than one polynucleotide may encode a discrete product, the term also include alleles and polymorphisms of a gene that encode the same product, or a functionally associated (including gain, loss, or modulation of function) analog thereof.

The term “gene expression” refers to the process by which RNA and proteins are made from the instructions encoded in genes. Gene expression includes transcription and/or translation of nucleic acid material. The term “RNA transcript” refers to the product resulting from transcription of a DNA sequence. When the transcript is the original, unmodified product of a RNA polymerase catalyzed transcription, it is referred to as the primary transcript. An RNA transcript that has been processed (e.g., spliced, etc) will differ in sequence from the primary transcript. A processed RNA transcript that is translated into protein is often called a messenger RNA (mRNA). The term “messenger RNA or mRNA” refers to a form of RNA that serves as a template to direct protein biosynthesis. Typically, the amount of any particular type of mRNA (i.e., having the same sequence, and originating from the same gene) represents the extent to which a gene has been expressed. The term “complementary DNA or cDNA” refers to a DNA molecule that is complementary to mRNA. cDNAs can be made by DNA polymerase (e.g., reverse transcriptase) or by direct chemical synthesis. The term “complementary” refers to nucleic acid sequences that base-pair according to the standard Watson-Crick complementary rules, or that are capable of hybridizing to a particular nucleic acid segment under relatively stringent conditions. Nucleic acid polymers are optionally complementary across only portions of their entire sequences.

The terms “protein”, “polypeptide” and “peptide” are used herein interchangeably, and refer to amino acid sequences of a variety of lengths, either in their neutral (uncharged) forms or as salts, and either unmodified or modified by glycosylation, side chain oxidation, or phosphorylation. In certain embodiments, the amino acid sequence is a full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains such as oxidation of sulfhydryl groups. Thus, the term “protein” (or its equivalent terms) is intended to include the amino acid sequence of the full-length native protein or a fragment thereof, subject to those modifications that do not significantly change its specific properties. In particular, the term “protein” encompasses protein iso forms, i.e., variants that are encoded by the same gene, but that differ in their pI or MW, or both. Such isoforms can differ in their amino acid sequence (e.g., as a result of alternative splicing or limited proteolysis), or in the alternative, may arise from differential post-translational modification (e.g., glycosylation, acylation, phosphorylation).

The terms “protein analog” and “protein homolog” are used herein interchangeably. They refer to a polypeptide that possesses a similar or identical function as the protein but need not necessarily comprise an amino acid sequence that is similar or identical to the amino acid sequence of the protein or a structure that is similar or identical to that of the protein. Preferably, in the context of the present invention, a protein analog or homolog has an amino acid sequence that is at least 80%, more preferably, at least about: 80%, 85%, 90%, 95%, 96%, 97%, 98% or 99%, identical to the amino acid sequence of the protein. In certain preferred embodiments, an analog or homolog of a biomarker of the invention has an amino acid sequence that is at least 80% identical or at least 85% identical to the amino acid sequence of the biomarker.

The term “homologous” (or “homology”), as used herein is synonymous with the term “identity” and refers to the sequence similarity between two polypeptide molecules or between two nucleic acid molecules. When a position in both compared sequences is occupied by the same base or same amino acid residue, then the respective molecules are homologous at that position. The percentage of homology between two sequences corresponds to the number of matching or homologous positions shared by the two sequences divided by the number of positions compared and multiplied by 100. Generally, a comparison is made when two sequences are aligned to give maximum homology. Homologous amino acid sequences share identical or similar amino acid sequences. Similar residues are conservative substitutions for, or “allowed point mutations” of, corresponding amino acid residues in a reference sequence residue. “Conservative substitutions” of a residue in a reference sequence are substitutions that are physically or functionally similar to the corresponding reference residue, e.g., they have a similar size, shape, electric charge, chemical properties, including the ability to form covalent or hydrogen bonds, or the like. Particularly preferred conservative substitutions are those fulfilling the criteria defined for “accepted point mutation” by Dayhoff et al. (“Atlas of Protein Sequence and Structure”, 1978, Nat. Biomed. Res. Foundation, Washington, D.C., Suppl. 3, 22: 354-352).

As used herein, the term “a reagent that specifically detects expression levels” refers to a reagent used to detect and determine the expression level of a gene (here the ITIH5 gene). Examples of suitable reagents include, but are not limited to, nucleic acid probes capable of specifically hybridizing to the gene of interest or mRNA transcripts thereof, PCR primers capable of specifically amplifying the gene of interest or mRNA transcripts thereof, and antibodies capable of specifically binding to the protein encoded by the gene of interest (here the ITIH5 gene).

The term “antibody”, as used herein, refers to any immunoglobulin (i.e., an intact immunoglobulin molecule, an active portion of an immunoglobulin molecule, etc.) that binds to a specific epitope. The term encompasses monoclonal antibodies and polyclonal antibodies. All derivatives (e.g., chimeric antibodies, humanized antibodies, single-chain antibodies) and fragments thereof (e.g., Fab, Fv, scFv and Fd fragments), which maintain specific binding ability, are also included in the term. The term also covers any protein (or fusion protein) having a binding domain, which is homologous or largely homologous to an immunoglobulin-binding domain. These proteins may be derived from natural sources, or partly or wholly synthetically produced.

The term “specific binding”, when used in reference to an antibody, refers to an antibody binding to a predetermined antigen. Typically, the antibody binds with an affinity of at least 1×10⁷M⁻¹, and binds to the predetermined antigen with an affinity that is at least two-fold greater than the affinity for binding to a non-specific antigen (e.g., BSA, casein).

The terms “labeled”, “labeled with a detectable agent” and “labeled with a detectable moiety” are used herein interchangeably. These terms are used to specify that an entity (e.g., an antibody) can be visualized, for example, following binding to another entity (e.g., a protein biomarker). Preferably, a detectable agent or moiety is selected such that it generates a signal which can be measured and whose intensity is related to the amount of bound entity. In array-based methods, a detectable agent or moiety is also preferably selected such that it generates a localized signal, thereby allowing spatial resolution of the signal from each spot on the array. Methods for labeling proteins and polypeptides are well known in the art. Labeled polypeptides (e.g., antibodies) can be prepared by incorporation of or conjugation to a label, that is directly or indirectly detectable by spectroscopic, photochemical, biochemical, immunochemical, electrical, optical, or chemical means, or any suitable means. Suitable detectable agents include, but are not limited to, various ligands, radionuclides, fluorescent dyes, chemiluminescent agents, microparticles, enzymes, colorimetric labels, magnetic labels, and haptens.

The term “treatment” is used herein to characterize a method that is aimed at (1) delaying or preventing the onset of a disease or condition; or (2) slowing down or stopping the progression, aggravation, or deteriorations of the symptoms of the condition; or (3) bringing about ameliorations or the symptoms of the condition; or (4) curing the condition. A treatment may be administered prior to the onset of the disease, for a prophylactic or preventive action. It may also be administered after initiation of the disease, for a therapeutic action.

The terms “approximately” and “about”, as used in reference to a number, generally include numbers that fall within a range of 10% in either direction of the number (greater than or less than the number) unless otherwise stated or otherwise evident from the context (except where such number would exceed 100% of a possible value).

### DETAILED DESCRIPTION OF CERTAIN PREFERRED EMBODIMENTS

As mentioned above, the present invention provides a biomarker, ITIH5, whose expression correlates with the state of the uterus of female subjects, or more specifically with the state of the endometrium of female subjects. Also provided are methods and kits for using this biomarker for detecting the receptivity state of the endometrium of a mammal to embryo implantation, for diagnosing infertility in a mammal, for the early diagnosis of pregnancy, for detecting the window of embryo implantation in the endometrium of a mammal, for in vitro fertilization of a mammal, and for diagnosing endometriosis.

### I—ITIH5 as a Biomarker of Endometrium State

In one aspect, the present invention provides the identity of a gene (the ITIH5 gene), whose expression at the genome, transcriptome and proteome levels is indicative of endometrium state, and more specifically of endometrium receptivity.

As used herein, the term “ITIH5” refers to the gene that encodes the protein ITIH5. ITIH5 is the most recently described member of the inter-α-trypsin inhibitor (ITI) protein family. The members of the ITI family are proteoglycans (ITIH-1 to −5) that are able to bind hyaluronic acid, frequently referred to as hyaluronic acid-binding proteins (HABPs) (Zhao et al., J. Biol. Chem., 1995, 270: 26657-26663; Bost et al., Eur. J. Biochem. 1998, 252: 339-346). This interaction with hyaluronic acid was initially deciphered in the cumulus oocyte complex in mice, and was shown to stabilize the extracellular matrix (ECM) (Chen et al., J. Biol. Chem., 1994, 269: 28282-28287). The ITIH5 protein was initially identified and described as a prognostic marker for breast cancer (Himmelfarb et al., Cancer Lett., 2004, 204: 69-77; Veeck et al., Oncogene 2008: 27:865-76). More recently, it has also been shown to be downregulated in bladder cancer (Lu et al., Am. J. Transl. Res., 2010, 3: 8-27) and poorly differentiated thyroid carcinomas (Pita et al., Br. J. Cancer, 2009, 101: 1782-1791).

In embodiments concerning a particular mammal species, the term “ITIH5” refers to the gene of that particular mammal species, and the term “ITIH5” prefers to a protein encoded by the gene. The ITIH5 gene of a variety of mammal species and corresponding ITIH5 protein have been sequenced and are known in the art.

In embodiments concerning a human subject, the term “ITIH5” refers to the human ITIH5 gene that is located on the small (p) arm of chromosome 10 at position 14 (Gene ID: 80760) and that encodes the protein ITIH5 (inter-alpha-trypsin inhibitor 5). Three isoforms of the mRNA product are known (isoform (1): GenBank NM_{—}030569.6 or SEQ ID NO: 1; isoform (2): GenBank NM_{—}032817.5 or SEQ ID NO: 2; and isoform (3): GenBank NM_{—}001001851.2 or SEQ ID NO: 3) which correspond to three isoforms of the protein ITIH5. The three isoforms of the protein ITIH5 are: inter-alpha-trypsin inhibitor heavy chain H5 isoform 1 precursor (GenBank NP_{—}085046.5 or SEQ ID NO: 4, which is the longest isoform); inter-alpha-trypsin inhibitor heavy chain H5 isoform 2 (GenBank NP_{—}116206.4 or SEQ ID NO: 5, which has an alternate and shorter N-terminus, compared to isoform 1); and inter-alpha-trypsin inhibitor heavy chain H5 isoform 3 precursor (GenBank NP_{—}001001851.1 or SEQ ID NO: 6, which has an alternative and shorter C-terminus, compared to isoform 1).

The present inventors have found that the female genital tract of mouse embryo expresses a 105 kDa isoform of the ITIH5 protein, which corresponds to the expected size of the ITIH5 precursor (isoform 1) and a lower molecular weight (about 50 kDa) isoform of the ITIH5 protein, which corresponds to isoform 3 (or embryonic isoform). The uterus of adult mice (whether pregnant or not) expresses isoform 1 and the mature form of the ITIH5 protein (about 75 kDa), which corresponds to isoform 2 (or adult isoform).

In humans, the three isoforms of the protein ITIH5 share a common domain having the amino acid sequence set forth in SEQ ID NO: 7. Isoform 2 of the ITIH5 protein is characterized by the domain having the amino acid sequence set forth in SEQ ID NO: 8, while isoform 3 of the ITIH5 protein is characterized by the domain having the amino acid sequence set forth in SEQ ID NO: 9.

Using the above sequence information, it is within the capabilities of one skilled in the art to develop reagents that specifically detect the expression levels of all the isoforms or that distinguish between isoform 2 and isoform 3 (i.e., between the adult and embryonic isoforms). Such reagents may be, in particular, antibodies capable of specifically binding to the protein products. An antibody useful in the practice of methods of the present invention may be an antibody that specifically binds to all three isoforms of the ITIH5 protein (i.e., that specifically binds to an epitope comprised in SEQ ID NO: 7), or an antibody that specifically binds to isoform 2 of the ITIH5 protein (i.e., that specifically binds to an epitope comprised in SEQ ID NO: 8), or an antibody that specifically binds to isoform 3 of the ITIH5 protein (i.e., that specifically binds to an epitope comprised in SEQ ID NO: 9). In embodiments wherein endometrium receptivity is concerned, the antibody specifically binds to all three isoforms of the ITIH5 protein or to isoform 2 of the ITIH5 protein. In embodiments wherein endometriosis is concerned, the antibody specifically binds to isoform 3 of the ITIH5 protein.

The invention also encompasses these antibodies and their use in the methods of the present invention.

One skilled in the art also knows how to develop reagents that specifically detect the expression levels of the ITIH5 gene of other mammal species, at the nucleic acid level or at the protein level. Since the ITIH5 gene is conserved in many species (including, but not limited to, human, chimpanzee, dog, cow, and mouse), the reagents that specifically detect the ITIH5 expression levels may be designed to be usable for more than one mammal species.

### II—Detection of the Biomarker ITIH5

In the methods of the present invention, the level of ITIH5 expression can be assessed at the protein or nucleic acid level. Method for determining the level of expression of a gene at either the nucleic acid or protein level are well known in the art and include, but are not limited to, immunoblots (western blots), northern blots, Southern blots, enzyme linked immunosorbent assay (ELISA), immunoprecipitation, immunofluorescence, flow cytometry, immunohistochemistry, nucleic acid hybridization techniques, nucleic acid reverse transcription methods, and nucleic acid amplification methods.

### Biological Samples

The methods described herein may be applied to the testing of any biological sample allowing ITIH5 to be assayed.

Thus, in certain embodiments, the biomarker is detected in a sample of endometrial tissue. However, in certain preferred embodiments of the present invention, the biomarker is detected in a sample of biological fluid selected from the group consisting of blood (e.g., whole blood, serum or plasma), urine, uterine/cervical secretions, and vaginal secretions. Preferably, the biomarker ITIH5 is detected in blood, serum or urine.

In certain embodiments, the inventive methods are performed on the biological sample without any major manipulation of the sample. In other embodiments, the inventive methods are performed on nucleic acid extracts or protein extracts prepared from the biological sample.

For example, RNA may be extracted from endometrial tissue samples and analyzed using a method of the invention. Methods of RNA extraction are well known in the art (see, for example, J. Sambrook et al., “Molecular Cloning: A Laboratory Manual”, 1989, 2^{nd} Ed., Cold Spring Harbour Laboratory Press: New York). Most methods of RNA isolation from bodily fluids or tissues are based on the disruption of the tissue in the presence of protein denaturants to quickly and effectively inactivate RNases. Generally, RNA isolation reagents comprise, among other components, guanidium thiocyanate and/or beta-mercaptoethanol, which are known to act as RNase inhibitors. Isolated total RNA may then be further purified from the protein contaminants and concentrated by selective ethanol precipitations, phenol/chloroform extractions followed by isopropanol precipitation (see, for example, P. Chomczynski and N. Sacchi, Anal. Biochem., 1987, 162: 156-159) or cesium chloride, lithium chloride or cesium trifluoroacetate gradient centrifugations.

Numerous different and versatile kits can be used to extract RNA (i.e., total RNA or mRNA) from human bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Giagen, Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

In certain embodiments, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2^{nd} Ed., Cold Spring Harbour Laboratory Press: New York; “Short Protocols in Molecular Biology”, F. M. Ausubel (Ed.), 2002, 5^{th} Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase).

The inventive methods may also be performed on a protein extract from the biological sample. Preferably, the protein extract contains the total protein content. Methods of protein extraction are well known in the art (see, for example “Protein Methods”, D. M. Bollag et al., 2^{nd} Ed., 1996, Wiley-Liss; “Protein Purification Methods: A Practical Approach”, E. L. Harris and S. Angal (Eds.), 1989; “Protein Purification Techniques: A Practical Approach”, S. Roe, 2^{nd} Ed., 2001, Oxford University Press; “Principles and Reactions of Protein Extraction, Purification, and Characterization”, H. Ahmed, 2005, CRC Press: Boca Raton, Fla.). Different kits can be used to extract proteins from bodily fluids and tissues that are commercially available from, for example, BioRad Laboratories (Hercules, Calif.), BD Biosciences Clontech (Mountain View, Calif.), Chemicon International, Inc. (Temecula, Calif.), Calbiochem (San Diego, Calif.), Pierce Biotechnology (Rockford, Ill.), and Invitrogen Corp. (Carlsbad, Calif.). User Guides that describe in great detail the protocol to be followed are usually included in all these kits. Sensitivity, processing time and costs may be different from one kit to another. One of ordinary skill in the art can easily select the kit(s) most appropriate for a particular situation. After the protein extract has been obtained, the protein concentration of the extract is preferably standardized to a value being the same as that of the control sample in order to allow signals of the protein markers to be quantified. Such standardization can be performed using photometric or spectrometric methods or gel electrophoresis.

### Determination of ITIH5 Protein Expression Levels

In certain embodiments, ITH5 expression levels are determined at the protein level.

Determination of protein expression levels in the practice of the inventive methods may be performed by any suitable method known in the art (see, for example, E. Harlow and A. Lane, “Antibodies: A Laboratories Manual”, 1988, Cold Spring Harbor Laboratory: Cold Spring Harbor, N.Y.).

Binding Agents.

In general, the expression level of a protein in a biological sample obtained from a subject or patient is determined by contacting the biological sample with a binding agent specific for the protein biomarker; determining, in the biological sample, the level of protein that binds to the binding agent; and comparing the protein level determined in the biological sample with the protein level measured in a control sample and/or with the level of one or more referenced proteins. As used herein, the term “binding agent” refers to an entity such as a polypeptide or antibody that specifically binds to an inventive protein biomarker. An entity “specifically binds” to a protein if it reacts/interacts at a detectable level with the protein but does not react/interact with polypeptides containing unrelated sequences or sequences of different polypeptides.

In the context of the present invention, the binding agent may be specific for one of the three isoforms of ITIH5, or specific for either the adult isoform (isoform 2) or the embryonic isoform (isoform 3). In many embodiments of the present invention, the binding agent is specific of the adult isoform.

In certain embodiments, the binding agent is a ribosome, with or without a peptide component, an RNA molecule, or a polypeptide (e.g., a polypeptide that comprises an amino acid sequence of a protein biomarker, a variant thereof, or a non-peptide mimetic of such sequence).

In other embodiments, the binding agent is an antibody specific for the biomarker of the invention (i.e. specific for the three isoforms of ITIH5 or specific of the adult isoform (or isoform 2) or specific of the embryonic isoform (or isoform 3)). Suitable antibodies for use in methods of the invention include monoclonal and polyclonal antibodies, immunologically active fragments (e.g., Fab or (Fab)₂ fragments), antibody heavy chains, humanized antibodies, antibody light chains, and chimeric antibodies. Antibodies, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985, 81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982, 299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czernik et al., Neuroprotocols, 1995, 6: 56-61; H. Zhang et al., J. Biol. Chem. 2002, 277: 39379-39387; S. L. Morrison et al., Proc. Natl. Acad. Sci., 1984, 81: 6851-6855; M. S, Neuberger et al., Nature, 1984, 312: 604-608; S. Takeda et al., Nature, 1985, 314: 452-454). Antibodies to be used in the methods of the invention can be purified by methods well known in the art (see, for example, S. A. Minden, “Monoclonal Antibody Purification”, 1996, IBC Biomedical Library Series: Southbridge, Mass.). For example, antibodies can be affinity-purified by passage over a column to which a protein biomarker of the invention, or fragment thereof, is bound. The bound antibodies can then be eluted from the column using a buffer with a high salt concentration.

Instead of being prepared, antibodies to be used in the methods of the present invention may be obtained from scientific or commercial sources. Examples of commercially available anti-ITIH5 antibodies include, but are not limited to, the anti-ITIH5 antibody produced in rabbit from Sigma-Aldrich, LifeSpan BioSciences, Abcam, Abgent, Atlas Antibodies, Acris Antibodies GmbH, Thermo Scientific Pierce Antibodies, Uscn LifeScience Inc. or Novus Biologicals.

Labeled Binding Agents.

Preferably, the binding agent (e.g., antibody) is directly or indirectly labeled with a detectable moiety. The role of a detectable agent is to facilitate the detection step by allowing visualization of the complex formed by reaction or association between the binding agent and the protein biomarker. Preferably, the detectable agent is selected such that is generates a signal which can be measured and whose intensity is related (preferably proportional) to the amount of protein biomarker present in the sample being analyzed. Methods for labeling biological molecules such as polypeptides and antibodies are well-known in the art (see, for example, “Affinity Techniques. Enzyme Purification: Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors, and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens, and proteins for which antisera or monoclonal antibodies are available.

In certain embodiments, the binding agent (e.g., an antibody) may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex, Sepharose, liposomes, carboxymethyl cellulose, polyacrylamydes, polystyrene, gabbros, filter paper, magnetite, ion-exchange resin, plastic film, plastic tube, glass, polyamine-methyl vinyl-ether-maleic acid copolymer, amino acid copolymer ethylene-maleic acid copolymer, nylon, silk, and the like. A binding agent may be indirectly immobilized using a secondary binding agent specific for the first binding agent (e.g., a mouse antibody specific for a protein biomarker may be immobilized using an sheep anti-mouse IgG Fc fragment specific antibody coated on the carrier or support). The secondary binding agent may be coupled to a detectable tag, such as for example, an enzyme, fluorophore, or chromophore.

Such antibodies can be used to determine ITIH5 expression levels in various immunoassays. Examples of such assays are radioimmunoassay, enzyme immunoassays (e.g., ELISA), immunofluorescence, immunoprecipitation, latex agglutination, hemagglutination, and histochemical tests, which are conventional methods well-known in the art. As will be appreciated by one skilled in the art, the immunoassay may be competitive or non-competitive. Methods of detection and quantification of the signal generated by the complex formed by reaction or association of the binding agent with the protein biomarker will depend on the nature of the assay and of the detectable moiety (e.g., fluorescent moiety).

### Determination of ITIH5 Nucleic Acid Expression Levels

In certain embodiments, ITH5 expression levels are determined at the nucleic acid level.

Nucleic acid-based techniques for assessing expression are well known in the art and include, for example, determining the level of ITIH5 mRNA in a biological sample (e.g., endometrial tissue sample). Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length, preferably between 15 and 40 nucleotides in length, and sufficient to specifically hybridize under stringent conditions to an mRNA or genomic DNA encoding ITIH5.

A nucleic acid probe may be labeled with a detectable moiety, as mentioned above. The association between the nucleic acid probe and detectable moiety can be covalent or non-covalent. Detectable moieties can be attached directly to the nucleic acid probes or indirectly through a linker (E. S. Mansfield et al., Mol. Cell. Probes, 1995, 9: 145-156). Methods for labeling nucleic acid molecules are well-known in the art (for a review of labeling protocols, and label detection techniques, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153).

### Expression Levels of ITIH5

Once the ITIH5 expression level has been determined (for example as described above) for the biological sample obtained from a subject tested, it may be compared to the ITIH5 expression level(s) determined in one or more control or reference samples (see below).

As known in the art, comparison of expression levels according to methods of the present invention is preferably performed after the expression levels obtained have been corrected for both differences in the amount of sample assayed and variability in the quality of the sample used (e.g., volume of sample, amount of protein extracted or number of cells stained, or amount and quality of mRNA tested). Correction may be carried out using any suitable method well-known in the art. For example, the protein concentration of a sample may be standardized using photometric or spectrometric methods or gel electrophoresis or via cell counting before the sample is analyzed. For analyses performed on nucleic acid molecules, correction may be carried out by normalizing the levels against reference genes (e.g., housekeeping genes such as, for example, the B2M (β-2-microglobulin) gene and the HPRT1 (hypoxanthine phosphoribosyltransferase) gene) in the same sample.

Normalized expression levels of the ITIH5 biomarker determined in a biological sample to be tested according to a method of the invention may then be compared to the normalized expression levels of the ITIH5 biomarker determined in one or more control biological samples.

In the context of the present invention, the term “control”, when used to characterize a subject, or a biological sample obtained from a subject, refers to a subject, or a biological sample obtained from a subject, that is healthy and exhibiting normal endometrium properties (e.g., normal endometrium receptivity, normal window of implantation) and undergoing a normal endometrium-associated event (e.g., normal menstrual or estrous cycle, normal embryo implantation, or normal pregnancy). The subject to be tested by a method of the invention and the control subject must be of the same sex and same mammalian species. The terms “normal” and “healthy” are used herein interchangeably to qualify an endometrium property or endometrium-associated event that is not associated with a pathology and/or that is observed in the majority of healthy subjects.

Preferably, normalized expression levels of the ITIH5 biomarker determined in a biological sample to be tested according to a method of the invention are compared to the average of normalized expression levels of the ITIH5 biomarker determined for biological samples obtained from a significant number of control subjects.

The term “control” also refers to a subject, or a biological sample obtained from a subject, that has been diagnosed with an abnormal endometrium property (e.g., defective endometrium receptivity, shorter window of implantation than normal or longer window of implantation than normal) or with an abnormal endometrium-associated disease or condition (e.g., abnormal menstrual or estrous cycle, defective embryo implantation, infertility, pathological pregnancy, pregnancy defects, miscarriage, or endometriosis).

Comparison of ITIH5 expression level(s) determined for one or more biological samples obtained from the subject to be tested with reference values, as described above, allows to establish a correlation between the expression level(s) measured and an endometrium property (e.g., normal endometrium receptivity, defective endometrium receptivity, normal window of implantation, shorter window of implantation than normal or longer window of implantation than normal) or an endometrium-related event or condition (e.g., normal menstrual or estrous cycle, normal embryo implantation, or normal pregnancy, abnormal menstrual or estrous cycle, defective embryo implantation, infertility, pathological pregnancy, pregnancy defects, miscarriage, or endometriosis).

One skilled in the art knows how to select the controls and comparisons to be performed to reach the endometrium-related prognosis or diagnosis.

### III—Uses of the Inventive Methods

As will be appreciated by those of ordinary skill in the art, a biomarker whose temporal expression during the estrous/menstrual cycle and during gestation correlates with the state (normal or pathological) of the endometrium can be used to characterize biological samples of subjects and patients, and to provide a prognosis or diagnosis regarding an endometrium-related disease or condition. Thus, using a method of the invention, the endometrium may be monitored for embryo receptivity and embryo implantation. ITIH5 can also be used for the early diagnosis of endometriosis.

### Endometrium Receptivity and Window of Receptivity

In one embodiment of the present invention, ITIH5 is used as a biomarker in which higher levels of ITIH5 protein or mRNA during the menstrual/estrous cycle are correlated with increased endometrial receptivity to embryo implantation or to greater endometrial maturation in female mammals. The highest levels ITIH5 protein or mRNA corresponds to the window of receptivity to embryo implantation.

The present invention provides a method for detecting endometrial receptivity to embryo implantation in a female mammal. The method for detecting endometrial receptivity to embryo implantation comprises steps of: (a) detecting the level of expression of ITIH5 in a biological sample obtained from the female mammal; and (b) correlating the level of expression of ITIH5 in the biological sample with endometrial receptivity to embryo implantation. In some embodiments, the method comprises detecting the level of expression of ITIH5 in biological samples obtained from a plurality of stages of the menstrual or estrous cycle of the female mammal.

The present invention also provides a method for detecting the window of embryo implantation in a female mammal. The method for detecting the window of embryo implantation of in a female mammal comprises steps of: (a) providing or obtaining a biological sample from said female mammal; (b) measuring the expression level of ITIH5 in the biological sample obtained from the female mammal; (c) repeating steps (a) and (b) with biological samples obtained from a plurality of stages of the menstrual or estrous cycle of said female mammal; and (d) determining the period of time of the menstrual or estrous cycle when the endometrium of said female subject is available for embryo implantation, based on the levels of expression measured.

As described in more detail below, the method for detecting endometrial receptivity to embryo implantation in a female mammal and/or the method for detecting the window of embryo implantation in a female mammal may be used for diagnosing infertility in a female mammal, or for monitoring the effects of a treatment protocol to induce ovarian hyperstimulation or ovulation induction on endometrium receptivity in a female mammal, or for assisted reproduction in a female mammal, or for assessing the probability of success of embryo implantation in the endometrium of a female mammal following a naturally achieved conception or a conception resulting from assisted reproduction technology, or for diagnosing pregnancy in a female mammal, or for the early diagnosis of endometriosis in a female mammal.

### Diagnosis of Infertility, Defective Endometrium Receptivity or Altered Endometrium Receptivity

Determination of endometrial receptivity and/or of the window of implantation allows infertility due to defective or altered endometrial receptivity to be diagnosed. A defective or altered endometrium receptivity may be characterized by levels of ITIH5 expression that are lower than normal or by a temporal expression of ITIH5 that is different from the normal temporal expression or by a window of implantation that is shorter or longer than normal (see above the definition of ‘normal’) or by any combination thereof.

Thus, the present invention provides a method for diagnosing infertility in a female placental mammal, comprising steps of: (a) providing or obtaining a biological sample from said female mammal; (b) measuring the expression level of ITIH5 in the biological sample obtained from the female mammal; and (c) correlating the level of expression of ITIH5 in the biological sample with infertility in said female mammal. In certain embodiments, the method for diagnosing infertility comprises detecting the level of expression of ITIH5 in biological samples obtained from a plurality of stages of the menstrual or estrous cycle of the female mammal. In certain embodiments, the method for diagnosing infertility comprises determining the period of time of the menstrual or estrous cycle when the endometrium of said female mammal is available for embryo implantation, based on the levels of expression measured.

Such a method may be used to diagnose women who repeatedly fail to become pregnant and who have no other apparent etiology for their infertility. Such a method may also be used to diagnose women who suffer from recurrent miscarriage. Recurrent miscarriage is defined as three or more consecutive miscarriages. It is experienced by 1% to 2% of couples that try to conceive.

A method according to the invention may, for example, be used to diagnose luteal phase dysfunction (LPD). LPD is characterized by developmental delay of the endometrium and occurs when the luteal phase is shorter than normal. It is a known cause of infertility, because of dyssynchrony between the fertilized egg and the endometrium. If an embryo is ready to attach but the endometrium is delayed, then pregnancy is not likely to occur. The causes for LPD include inadequate hormonal output by the ovary, and may implicate defective signaling from higher centers such as inadequate gonadotropic hormone output from the pituitary or hypothalamus. LPD is a known cause of infertility and spontaneous abortion. LPD can be corrected with hormone augmentation. When LPD is diagnosed using a method according to the invention, the method may further comprise a step of prescribing an appropriate hormone treatment and/or administering an appropriate hormone treatment.

Increasing evidence suggest that some women may experience recurrent miscarriage when “super-active” endometrium allows embryos of low viability to implant, presenting a clinical pregnancy before miscarrying. Super-fertility is characterized by an endometrium that is more conducive for implanting embryos, both healthy and unhealthy ones. Super-fertility may result from a window of implantation being extended (i.e., longer than normal) (Salker et al., 2010, PLoS One 5: e10287), thus reducing the ability of the decidualized endometrium to be ‘selective’ in response to embryo quality. A method of the invention may therefore be used for diagnosing infertility due to “super-fertility”.

Using methods described herein, skilled physicians may, based on the diagnosis reached, select and prescribe treatments adapted to each patient. Selection of an appropriate therapeutic regimen for a given patient may be made based solely on the diagnosis provided by the inventive methods. Alternatively, the physician may also consider other clinical or pathological parameters used in existing methods to diagnose infertility.

Determination of endometrial receptivity also allows diagnosis of alteration of endometrial receptivity due to therapeutic treatments administered to the subject. In particular, it is known that ovarian hyperstimulation and ovulation induction can alter the natural course of endometrial development.

Thus, the present invention provides a method for monitoring the effects of a treatment protocol to induce ovarian hyperstimulation or ovulation induction on endometrium receptivity in a female mammal, comprising steps of: (a) providing or obtaining a biological sample from said female mammal; (b) measuring the expression level of ITIH5 in the biological sample obtained from the female mammal; and (c) detecting and/or identifying, in said female mammal, an alteration of the endometrium receptivity due to the treatment, based on the expression level measured. In certain embodiments, the method further comprises determining the period of time of the menstrual or estrous cycle when the endometrium of said female mammal is available for embryo implantation, based on the levels of expression measured.

### Assisted Reproduction

Determination of endometrial receptivity and/or of the window of implantation according to a method of the present invention may find utility in assisted reproduction technology, in particular in in vitro fertilization/embryo transfer procedures.

Thus, the present invention provides a method for assisted reproduction in a female placental mammal, comprising steps of: (a) measuring the expression level of ITIH5 in a biological sample obtained from the female mammal; (b) repeating step (a) with biological samples obtained from a plurality of stages of the menstrual or estrous cycle of said female mammal; (c) correlating the level of expression of ITIH5 in one or more samples of step (b) with endometrial maturation; and (d) transferring or introducing at least one embryo into the uterus of said female mammal when said endometrium is mature.

In certain embodiments, the embryo that is transferred develops from a zygote formed by the combination of an egg and sperm in vitro.

Using this method, embryo transfer can be timed to coincide with a receptive endometrium or can be delayed, cryopreserving embryos, until a more appropriate state of receptivity is attained.

In certain embodiments the method of assisted reproduction further comprises monitoring the transferred embryo for implantation (see below).

The method of assisted reproduction according to the present invention may be applied to women. However, in modern agriculture, assisted reproductive technologies are being used for enhancement of reproductive performance and genetic improvement. In addition, they can have substantial contribution in preservation of endangered species or breeds, as well as in eradication programs of diseases. Assisted reproductive technologies, such as estrus synchronization, estrus induction, synchronization of parturition, superovulation, in vitro fertilization, embryo transfert, multiple ovulation and embryo transfer (MOET) and artificial insemination, have been introduced to overcome reproductive problems, to increase the offspring from selected females, to reduce the generation interval in farm animals, to control diseases and to cut production costs.

Thus, in certain embodiment, the female mammal to which a method of assisted reproduction according to the present invention is applied is a mammalian farm animal (e.g., cattle, sheep), mammalian sport animal (e.g. race horse), and mammalian pet (e.g., cat, dog).

### Embryo Implantation

In one embodiment of the present invention, ITIH5 is used as a biomarker in which a peak of ITIH5 expression following naturally achieved conception or conception resulting from an assisted reproduction technology is correlated with implantation of embryo in female mammals, and subsequent increase of ITIH5 expression level is indicative of a higher probability of continued success of a pregnancy.

### Probability of Success of Implantation and of Continued Success of Pregnancy

The present invention provides a method for assessing the probability of success of embryo implantation in the endometrium of a female mammal following a naturally achieved conception or a conception resulting from assisted reproduction technology, comprising steps of: (a) measuring the expression level of ITIH5 in a biological sample obtained from the female mammal; and (b) correlating the level of expression of ITIH5 in said biological sample with the probability of success of embryo implantation in the endometrium of said female mammal.

In certain embodiments, the method comprises measuring the level of expression of ITIH5 in biological samples obtained from a plurality of stages of the menstrual or estrous cycle of the female subject.

### Early Diagnosis of Pregnancy

In another embodiment of the present invention, a method for the early diagnosis of pregnancy in women is provided. Indeed, most chemical tests for pregnancy look for the presence of the beta subunit of human chorionic gonadotropin (β-hCG) in the blood or urine. β-hCG can only be detected in urine or blood after implantation, which in humans occurs six to twelve days after fertilization. Since detection of a peak of ITIH5 expression according to the present invention following conception is indicative of implantation itself, pregnancy can be diagnosed earlier than with the β-hCG system.

Thus, a method is provided for diagnosing pregnancy in a woman comprises steps of: (a) detecting the level of expression of ITIH5 in a biological sample obtained from the female mammal (in particular a woman); and (b) correlating the level of expression of ITIH5 in the biological sample with embryo implantation and diagnosing pregnancy. In this method, the biological sample is preferably blood, serum or urine.

In certain embodiments, the method may further comprise a step of: detecting and/or measuring, in a biological sample of the female mammal, the expression level of one or more hormones selected from the group consisting of hormone folliculostimulante (FSH), luteinizing hormone (LH), progesterone, anti-Müllerian hormone (AMH), estradiol, beta-human chorionic gonadotropin (β-hCG) and any combination thereof.

### Early Diagnosis of Endometriosis

Endometriosis is one of the most common gynecological disorders, affecting up to 10-15% of women of reproductive age. It is mainly associated with severe pelvic pain and/or infertility, but other symptoms may include diarrhea, intestinal pain, painful intercourse, abdominal tenderness, cramping, back ache, menstrual cramps, and excessive menstrual bleeding. Endometriosis is characterized by the implantation and growth of endometrial cells (which normally constitute the lining of the uterus) in extra-uterine sites, most frequently in the peritoneal cavity. There is no cure for endometriosis, but it can be treated in a variety of ways, including pain medication, hormonal treatments, and surgery. Annual healthcare costs and costs of productivity loss associated with endometriosis have been estimated at $2801 and $1023 per patient, respectively (Simoens et al., Human Reprod. Update, 2007, 13(4): 395-404).

Endometriosis presents difficulties in diagnosis. Direct visualization of the endometriotic lesions under surgical procedures (laparoscopy or laparotomy) is currently the only reliable method to diagnose endometriosis. However, this method is highly invasive (as it involves surgery under general anesthesia) and costly. The period of time between the onset of symptoms and disease diagnosis can be as long as 8 to 12 years (Hadfield et al., Human Reproduction, 1996, 11(4): 878-880). Ideally, the prospect to diagnose endometriosis more easily, rapidly, and as early as possible during the course of the disease would definitely reduce the number of years during which patients endure pain, infertility or other symptoms.

Based on this perspective, several investigators have sought to identify biological markers (proteinic and genetic) that could efficiently be used as predictive tools for endometriosis. However, so far, no method has been able to accurately diagnose endometriosis with as high a degree of sensitivity than what has been documented with laparoscopy or laparotomy.

The present invention provides a method for the early diagnosis of endometriosis in female subjects, in particular in women of reproductive age. The method for diagnosing endometriosis comprises steps of: (a) measuring the level of expression of ITIH5 in a sample of biological fluid obtained from the subject; and (b) correlating the level of expression of ITIH5 in the biological sample with a diagnosis of endometriosis.

In this method, the step of measuring the level of expression of ITIH5 is performed using a reagent that specifically detects the embryonic isoform of ITIH5 (i.e., isoform 3). Indeed, the embryonic origin of the endometrial cells which, in endometriosis, appear and flourish outside the uterine cavity, are of embryonic origin and are therefore believed to express the embryonic form of ITIH5.

### IV—Kits and Pregnancy Tests

In another aspect, the present invention provides kits comprising materials useful for carrying out a prognostic/diagnostic method of the invention. The prognostic/diagnostic procedures described herein may be performed by analytical laboratories, research laboratories, practitioners and by patients themselves. The invention provides kits that can be used in these different settings.

Materials and reagents for performing a prognostic/diagnostic method of the present invention may be assembled together in a kit. In certain embodiments, an inventive kit comprises at least one reagent that specifically detects expression levels of the ITIH5 biomarker. Thus, in certain embodiments, a kit comprises at least one reagent that specifically detects the expression level of the ITIH5 gene at the nucleic acid level. In other embodiments, a kit comprises at least one reagent that specifically detects the expression level of the ITIH5 gene at the protein level.

A kit may further comprise instructions for using the kit to perform a prognosis/diagnosis according to a method of the invention. Each kit preferably comprises the reagents that render the procedure specific. Thus for detecting/quantifying the ITIH5 protein, the reagent that specifically detects the protein expression levels may be an antibody that specifically binds to the ITIH5 protein, as described above. For detecting/quantifying ITIH5 expression at the nucleic acid level, the reagent that specifically detects gene or mRNA expression levels may be a nucleic acid probe complementary to the polynucleotide sequence (e.g., cDNA or oligonucleotide) or a nucleic acid primer, as described above. The nucleic acid primers may or may not be immobilized on a substrate surface (e.g., an array).

In addition, an inventive kit may further comprise at least one reagent for the detection of a protein biomarker-antibody complex formed between an antibody included in the kit (i.e., an anti-ITIH5 antibody) and the protein biomarker (i.e., ITIH5) present in a biological sample obtained from a subject. Such a reagent may be, for example, a labeled antibody that specifically recognizes antibodies from the species tested (e.g., an anti-human IgG), as described above. If the antibodies are provided attached to the surface of an array, a kit of the invention may comprise only one reagent for the detection of biomarker-antibody complexes (e.g., a fluorescently-labeled anti-human antibody).

Depending on the procedure, the kit may further comprise one or more of: extraction buffer and/or reagents, amplification buffer and/or reagents, hybridization buffer and/or reagents, immunodetection buffer and/or reagents, labeling buffer and/or reagents, and detection means. Protocols for using these buffers and reagents to perform different steps of the diagnostic procedure may be included in the kit.

The reagents may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

Instructions for using the kit according to a method of the invention may comprise instructions for processing the biological sample obtained from the subject or patient, instructions for performing the test, and/or instructions for interpreting the results as well as a notice in the form prescribed by a governmental agency (e.g., FDA) regulating the manufacture, use or sale of pharmaceuticals or biological products.

The invention also relates to the use of a kit described above for detecting endometrial receptivity to embryo implantation in a female mammal, or for detecting the window of embryo implantation of in a female mammal, or for diagnosing infertility in a female placental mammal, or for monitoring the effects of a treatment protocol to induce ovarian hyperstimulation or ovulation induction on endometrium receptivity in a female mammal, or for assisted reproduction in a female mammal, or for assessing the probability of success of embryo implantation in the endometrium of a female mammal following a naturally achieved conception or a conception resulting from assisted reproduction technology, or for diagnosing pregnancy in a female mammal, or for the early diagnosis of endometriosis.

In yet another aspect, the present invention provides pregnancy test kits, in particular home pregnancy test kits. As used herein, the term “home pregnancy test kit” refers to a test kit that involves a patient being able to do the test at home, for example, a urine test which indicates a positive or negative result by a color change or other means such as a digital output. The home test is designed to be used by someone with no medical experience and as such the urine type tests are ideal. The home test kits are sensitive to the presence of ITIH5, in urine, and change color, or otherwise indicate, when above the threshold sensitivity to the ITIH5 is detected in the particular test.

In certain embodiments, a home pregnancy test kit of the invention is a test strip. The term “test strip”, as used herein, is a strip kind used for the purpose of placing urine on a particular spot which initiates an ITIH5 urine color or other indicator test. The test strip can also be a digital type where an indicator screen displays a message such as “pregnant” or “not pregnant” or “yes” or “no” instead of a simple color change. The strip tests are also called stick tests.

A home pregnancy test kit of the invention may comprise more than one strip associated to each other, wherein one strip is sensitive to the presence of ITIH5, as described above, and at least one other strip is sensitive to the presence of a hormone selected from the group consisting of FSH, LH, progesterone, AMH, estradiol, and β-hCG. In certain embodiments, the home pregnancy test kit comprises two strips, the first strip being sensitive to the presence of ITIH5 and the second strip being sensitive to the presence of β-hCG. The two strips may be physically associated to each other.

A home pregnancy test kit of the invention may comprise two or more test strips, a first test strip comprising a first ITIH5 home urine test of a first ITIH5 sensitivity and the second and subsequent test strips comprising at least one ITIH5 home urine test less sensitive to ITIH5 than the first ITIH5 home urine test.

These home pregnancy test kits allow one to test for pregnancy and then confirm that the pregnancy is active, ongoing and progressing. By the nature of the serial test strip product, one can test and know that a woman is above one threshold and below the other in the test strip and on subsequent testing is above both thresholds indicating increasing ITIH5 and thus an active viable pregnancy. The first test strip then would be the most sensitive. This would establish embryo implantation just as using the test strip by itself would do. However, at a desired interval from the first test strip, a second different test strip is used to assess success of continued pregnancy.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text is not intended to suggest that experiments were actually performed or data were actually obtained.

Some of the results presented below have been reported by the present Applicants in a scientific article (Morcel et al., Orphanet J. Rare Dis., Mar. 15, 2011, 6:9) and in a manuscript (Morcel et al., “Involvement of ITIH5, a Candidate Gene for Congenital Uterovaginal Aplasia (Mayer-Rokitansky-Kiister-Hauser Syndrome), in Female Genital Tract Development) accepted for publication to Gene Expression and scheduled to be published in May 2013. The content of each of these documents is incorporated herein by reference in its entirety.

### Example 1

### Results on Animals

### Materials and Methods

### Animals

Animals were used in compliance with European Commission guidelines and with the approval of the “Haut Conseil des Biotechnologies” (#5450), and of the “Direction Départementale des Services Vétérinaires” (#35-44). Female CD1-Swiss mice reared in the Experimental Animal Department of the University of Rennes (France) were used. For embryo staging, the morning on which a vaginal plug was first observed was designated embryonic day 0 (E0). Vaginal smears were used to stage the estrus cycle.

RT-PCR and q-PCR

RNA-Extraction and cDNA Synthesis.

Total RNA was extracted from tissue samples using the RNeasy® Mini kit (Qiagen S.A, Courtaboeuf, France), according to the manufacturer's protocol. Total RNA was reversed-transcribed using the Moloney Murine Leukemia Virus (M-MLV) reverse transcriptase (Promega, Charbonniére, France) to generate cDNA, which was subsequently used for RT-PCR and RT-quantitative PCR.

PCR Amplification.

Complementary DNAs were amplified with Taq DNA polymerase (GoTaq® DNA polymerase, Promega), according to standard procedures. Primers were designed using the Primer 3′ software that is available online. All the primers had a similar (60° C.) melting temperature (Tm). Various pairs of primers were designed to bind to various sites along the length of the cDNA sequence, to strengthen the results and to facilitate the detection of mRNA isoforms.

Reverse transcription-quantitative PCR (RT-qPCR).

The Power SYBR Green PCR master mix (Applied Biosystems, Villebon-sur-Yvette, France) was used for qPCR with an ABI Prism 7000 Sequence Detection System (Applied Biosystems), as recommended by the manufacturer. Each sample was tested in triplicate. The yield of each cDNA produced was analyzed using the comparative Ct (threshold cycle) method of qPCR. The values were normalized with respect to the Ct obtained for amplification of the internal standard, the Hprt1 gene (hypoxanthine phosphoribosyltransferase 1). The expression of this gene was considered to be stable and similar in all cell types.

Whole-Mount In Situ Hybridization (WISH).

Preparation of Samples.

Female genital tracts were dissected out from embryos at stages E16.5 and E18.5. Tissues were fixed by incubation overnight at 4° C. in 4% paraformaldehyde (PFA) in phosphate-buffered saline (PBS) and dehydrated in a series of methanol solutions of increasing concentration.

Synthesis of Riboprobes.

A 463 bp cDNA probe corresponding to exons 14 and 15 of the Itih5 gene was inserted into pSPT18 (Roche, Mannheim, Germany) The recombinant plasmid was linearized by digestion with EcoRI. The antisense riboprobe was synthesized from the linearized plasmid in the presence of digoxigenin-labeled UTP, with a DIG-RNA labeling kit (Roche) and the SP6 RNA polymerase promoter, according to the manufacturer's instructions. Sense riboprobe synthesis required linearization of the plasmid by HindIII digestion and the use of the T7 RNA polymerase promoter.

Hybridization.

Tissues were rehydrated and treated with proteinase K according to standard procedures. Hybridization was performed at 65° C. for 12 hours in hybridization buffer (50% formamide, 5×SSC pH 7, 100 μg ml⁻¹ tRNA, 100 μg ml⁻¹ sodium heparin, 0.5 M EDTA, 10% CHAPS, 20% Tween-20) to which 200 ng/ml of one of the sense or antisense riboprobes was added. Samples were washed and incubated overnight at 4° C. with anti-digoxigenin alkaline phosphatase Fab fragments (Roche), at a dilution of 1:5,000. They were then thoroughly washed and incubated with BM purple solution (Roche) for signal detection. Once optimal signal intensity had been achieved, the samples were washed in PBT (0.1% Tween 20 in PBS) supplemented with 1 mM EDTA and fixed in 4% PFA in PBS. They were then dehydrated in a series of methanol solutions of increasing concentrations and stored in 100% methanol at −20° C.

Western-Blot Analysis.

Protein Purification.

Proteins were extracted from samples of frozen (−80° C.) genital tracts and livers from E18.5 mouse embryos, and from the genital tracts from pregnant and non pregnant mice. Tissues were homogenized in F9 lysis buffer (0.1 M Tris pH 7.5, 0.15 M NaCl, 0.1% SDS, 1% sodium deoxycholate, 1% Triton) and sonicated on ice (4° C.) for 5 minutes. Lysates were centrifuged at 10,000×g for 5 minutes at 4° C., and the supernatants were collected in fresh tubes. Protein concentration in the supernatants was determined with a bicinchoninic acid protein assay kit (QuantiPro™ BCA assay, Sigma-Aldrich, Lyons, France), according to the manufacturer's protocol. Protein solutions were mixed with an equal volume of 2× Laemmli buffer containing 100 mM Tris-HCl pH 6.8, 20% glycerol, 3% SDS, 5% β-mercaptoethanol, heated for 5 minutes at 95° C. and stored at −20° C. until analysis.

Production of the GST-Itih5 Fusion Protein.

A pCMV-SPORT plasmid (Invitrogen, Cergy Pontoise, France) containing the full-length cDNA encoding mouse Itih5 was obtained from RZPD (ImaGenes Gmbh, Berlin, Germany) A cDNA fragment corresponding to amino-acids 219-952 of the Itih5 protein was inserted into pGEX-3× (GE Healthcare, Saclay, France) and the resulting recombinant plasmid was used to produce a GST-fusion protein, GST-Itih5. E. coli cells were grown at 37° C. in 10 ml of LB medium supplemented with ampicillin (50 μg ml⁻¹), until the culture reached an OD₆₆₀ of 0.8 (about 3 hours). Synthesis of the GST-Itih5 fusion protein was induced by adding isopropyl 1′-D thiogalactoside (IPTG) to a final concentration of 1 mM. The cells were incubated for a further three hours at 37° C. and then harvested by centrifugation at 2,000 g for 10 minutes at 4° C. Proteins were released by resuspending the wet cell pellets in Laemmli lysis buffer and subjecting them briefly to sonication. The proteins were denatured by heating for 5 minutes at 95° C. and then subjected to SDS-PAGE. The GST-Itih5 fusion protein was approximately 105 kDa in size, consistent with its deduced molecular weight.

Antibody Production.

A rabbit polyclonal anti-Itih5 antibody was generated at Eurogentec (Seraing, Belgium), with a synthesized peptide corresponding to amino-acids 475-490 of the mouse protein (SEQ ID NO: 10: YDEIRTPLLSDIRIDY).

Western-Blot Analysis.

Proteins were separated by electrophoresis in an 8.5% polyacrylamide gel supplemented with 0.1% SDS, in a MiniProtean II electrophoresis system (Bio-Rad, Ivry sur Seine, France). They were transferred onto a nitrocellulose membrane (Millipore S.A.S, Molsheim, France), which was then saturated by incubation in PBT supplemented with 5% non fat milk powder for 1 hour at room temperature. The membrane was subsequently incubated at 4° C., for 90 minutes with rabbit polyclonal anti-Itih5 antibody at a dilution of 1:500 and washed five times in PBT. It was then incubated with PBT supplemented with a secondary antibody (horseradish peroxidase (HRP)-conjugated goat anti-rabbit antibody) at a dilution of 1:20,000, for 1 hour at 4° C., and washed five times in PBS. Immunoreactive proteins were detected by chemiluminescence, with an ECL kit (Amersham ECL™ Western Blotting Detection kit, GE Healthcare).

### Results

### Itih5 Gene Expression in the Mouse Female Genital Tract During Development

### RT-PCR and RT-qPCR

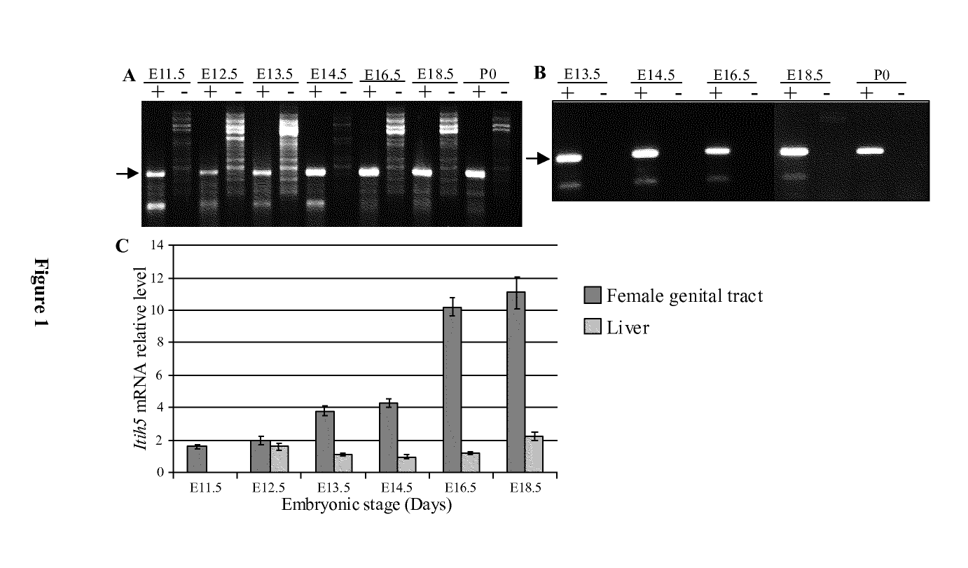

Itih5 mRNA expression was analyzed by RT-PCR between E11.5 and P0, in the female genital tract and in the liver. Itih5 was expressed in both tissues at each embryonic stage studied. Similar results were obtained with all primer pairs used (FIGS. 1A and 1B show RT-PCR products obtained with the Ex6-8 primers). RT-qPCR was used to measure changes in Itih5 mRNA levels during embryogenesis in the female genital tract and, for comparison, in the liver. The amount of Itih5 mRNA in the female genital tract increased considerably during embryogenesis, with relative levels increasing by a factor of about five between E11.5 and E18.5 (FIG. 1C). Furthermore, Itih5 mRNA was markedly more abundant in the female genital tract than in the liver, particularly after E16.5 (10 times higher): Itih5 gene expression remained stable, at low levels, throughout embryogenesis (FIG. 1C).

WISH Analysis.

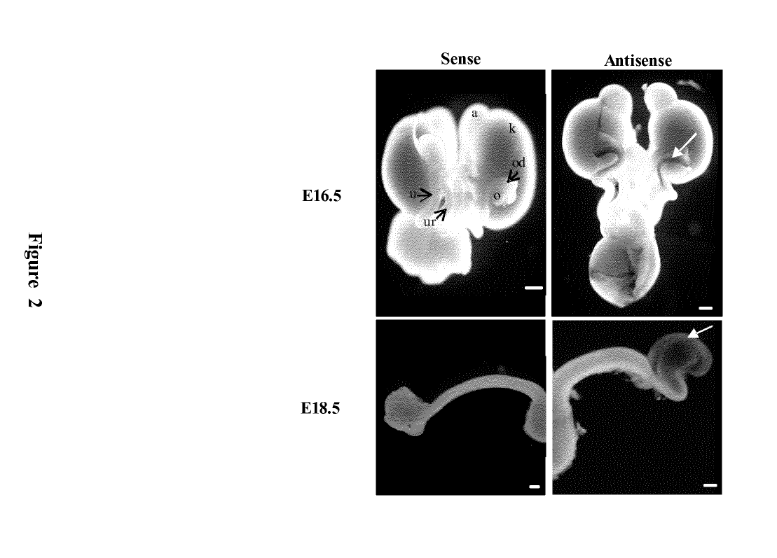

Whole-mount in situ hybridization was used at E16.5 and E18.5 to investigate the pattern of Itih5 gene expression in the female genital tract. Itih5 expression was clearly detected, at both embryonic stages, in the cranial part of the uterine horns (FIG. 2).

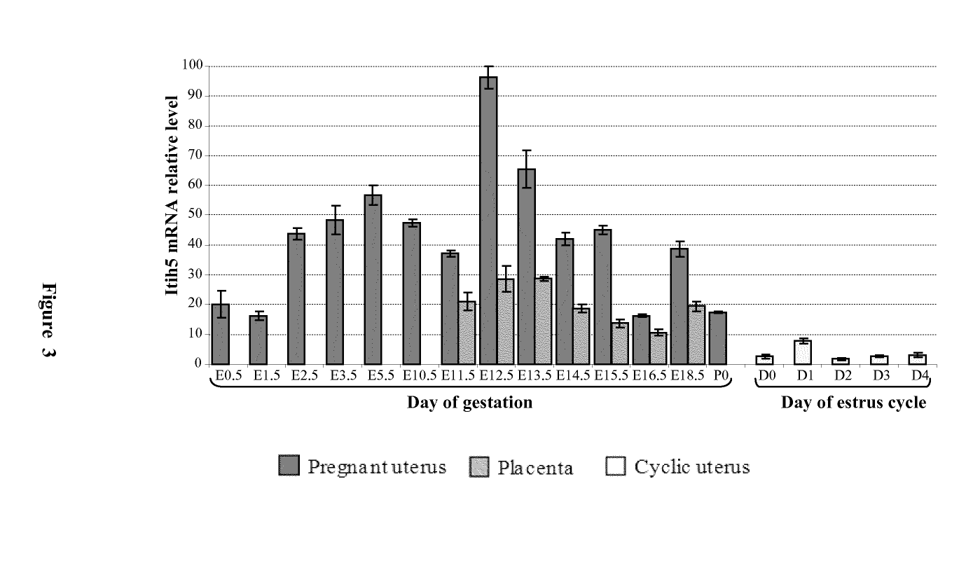

Itih5 Gene Expression in the Adult Mouse Uterus During Pregnancy and the Estrus Cycle, as Analyzed by RT-qPCR Analysis of Itih5 mRNA (FIG. 3)

Itih5 was found to be expressed in the uterus during gestation and during the estrus cycle. Itih5 mRNA was more abundant in the uterus of pregnant animals than in the placenta (1.5 to 4 times more abundant, depending on the stage of gestation). Itih5 mRNA levels in the uterus of pregnant mice peaked twice: one peak was at the E5.5 stage, with a larger peak observed between E12.5 and E13.5. By contrast, Itih5 mRNA was much less abundant in the cyclic uterus than in the placenta, with little variation during the course of the estrus cycle.

### The Genital Tract of Female Embryos and Adult Mice Produce Different Isoforms of ITIH5 Protein

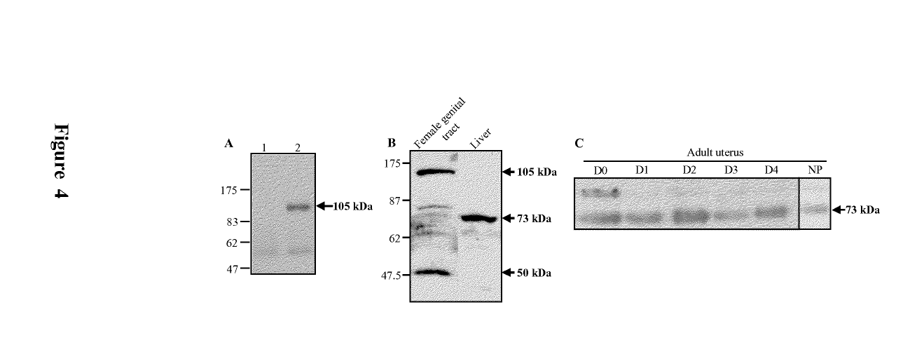

The specificity of the polyclonal antiserum was checked by western-blot experiments with the GST-Itih5 fusion protein (FIG. 4A). The Itih5 protein was studied by western blotting, at E18.5, in both female genital tract and liver samples, with the anti-Itih5 antibody used at a dilution of 1:500 (FIG. 4B). Two strong, specific bands were detected for the female genital tract of mouse embryos: a 105 kDa band corresponding to the expected size of the Itih5 precursor and a lower molecular weight (about 50 kDa) band possibly resulting from tissue-specific post-translational processing and corresponding to an isoform smaller than the 73 kDa form found in the liver (FIG. 4B). By contrast, only the mature form of Itih5 protein (73 kDa) was detected in the uterus of adult mice, whether or not they were pregnant (FIG. 4C).

### Discussion

The Itih5 gene was found to be expressed in the mouse uterus during pregnancy and during female genital tract development. Furthermore, whereas endometrial levels of expression of the Itih-1 to -4 genes remain constant throughout all stages of the estrus cycle and early pregnancy (Geisert et al., Reproduction 2003; 126:621-7), the results obtained clearly demonstrate that levels of Itih5 gene expression in the female genital tract vary considerably with the physiological state of the uterus.

Itih5 mRNA was much more abundant in the uterus of pregnant adult mice than in the uterus of adult mice that were not pregnant. Interestingly, the present inventors found that the level of Itih5 expression in the uterus of pregnant mice was up to 50 times the maximal levels observed during the estrus cycle. This strongly suggests that Itih5 plays a key role during gestation, probably in implantation and uterus growth, as previously shown for other ECM components (Shynlova et al., Biol Reprod 2004; 70:986-92). In particular, two peaks of expression were observed when the embryo was at stages E5.5 and E12.5, corresponding to specific stages of pregnancy in the mouse. Indeed, the period around E5.5 is regarded as the “implantation window”: it corresponds to the physiological preparation of the endometrium for implantation and coincides with the arrival of the embryo for implantation (Cross et al., Science 1994; 266:1508-18). During this period, uterine cells proliferate and/or differentiate in a particular spatiotemporal manner (Cross et al., Science 1994; 266:1508-18). Furthermore, implantation seems to be regulated locally by interactions involving ECM components (Wang et al., Cells Tissues Organs 2002; 172:190-201; Armant et al., Semin Reprod Med 2000; 18:273-87). Invasive trophoblasts adhere to, spread and migrate on ECM substrates and penetrate three-dimensional ECM structures (Dey et al., Endocr Rev 2004; 25:341-73). Following implantation, the uterus grows significantly from stage E12.5 onwards. Indeed, pregnancy involves the growth and differentiation of myometrial cells (Young, Ann NY Acad Sci 2007; 1101:72-84) and myometrial growth occurs mostly after mid-gestation (corresponding to stage E12.5 in mice) (Shynlova et al., Reproduction 2010; 139:247-53). In humans, the uterus increases in weight from about 50 g in the non-pregnant state to about 1200 g at term, thus increasing in mass by a factor of 24 during the course of pregnancy (Johansson, Hypertension 1984; 6:11164-8). This myometrial growth is dependent on an increase in the synthesis of various ECM proteins, including collagens (type I, III and IV), elastin, fibronectin and laminin β2; each of these ECM components displays a specific temporal pattern of expression during gestation (Shynlova et al., Biol Reprod 2004; 70:986-92). The considerable variation of Itih5 mRNA levels in the mouse uterine during the course of gestation is particularly interesting in this respect. The ITIH5 protein thus appears to be one of the set of ECM proteins involved in myometrial growth.

The Itih5 gene products were also analyzed in embryogenesis and adult life. A 73 kDa protein was found in the adult uterus, during both the estrus cycle and pregnancy. This molecule seems to correspond to the mature form of the protein. Indeed, only one form of the mouse Itih5 protein (NP_{—}766059.1) is currently found in databases. This form has a sequence very similar to that of the human NP_{—}85046 isoform, corresponding to a polypeptide precursor of 952 amino-acid residues (105 kDa). This precursor undergoes posttranslational processing, including a trimming of the N-terminal end, with the removal of 18 amino-acid residues (signal peptide), and of the C-terminal end at the conserved cleavage site (DDPHFVV), resulting in the removal of 271 amino-acid residues (Himmelfarb et al., Cancer Left 2004; 204:69-77). The final mature protein is approximately 73 kDa in size and, therefore, corresponds to the protein observed in the adult mouse.

The Itih5 expression was also studied in the mouse female genital tract during development, from E11.5 onwards, because the Müllerian ducts are known to arise in the mesonephros at E11.5, subsequently differentiating into the female genital tract (Kobayashi et al., Development 2004; 131:539-49; Orvis et al., Dev Biol 2007; 306:493-504). The expression of this gene increased with the progression of genital tract development, from E11.5 to birth. Furthermore, Itih5 expression was markedly stronger in the developing female genital tract than in the liver at the same stage, even though the liver is the main source of ITIH proteins (Saguchi et al., J Biochem 1995; 117:14-8; Chan et al., Biochem J 1995; 306 (Pt 2):505-12; Salier et al., Biochem J 1993; 296 (Pt 1):85-91). These observations suggest that ITIH5 plays a major role in the development of the female genital tract. A protein of about 50 kDa in size was detected during the differentiation and development of the Müllerian ducts, suggesting the presence of another Itih5 isoform not previously described in the mouse. This isoform may be equivalent to another known human isoform, NP_{—}116206, which is about 52 kDa in size after post-translational modifications. These observations are consistent with the known balance and combination of proteins forming the ECM, which may be highly variable and tissue-specific (Gorski et al., Curr Opin Cell Biol., 1998, 10: 586-93), resulting from the use of alternative promoters, diverse splicing patterns and various post-translational modifications (Gorski et al., Curr Opin Cell Biol., 1998, 10: 586-93).

The present study reports the temporal pattern of expression of the Itih5 gene in the female mouse genital tract during adulthood and embryogenesis. Two isoforms of Ithi5, of 73 and 50 kDa in size, in adult animals and embryos, respectively. Further investigations are required to elucidate the mechanisms of action of these two isoforms. These results indicate that ITIH5 probably plays a specific and major physiological role during cell development and differentiation in the female genital tract in mammals. These findings are consistent with the deletion of the human ITIH5 gene found in a case of MRKH syndrome (Morcel et al., Orphanet J Rare Dis 2011; 6:9), supporting the identification of this gene as a strong candidate gene for the syndrome.

### Example 2

### Results on Humans

Materials and Methods

### Subjects

Preliminary tests were performed on 160 female patients that were admitted in the gynecological emergency room of Rennes Hospital (Service de gynécologie, POGMR, CHU de Rennes, France), and 3 men.

### Determination of ITIH5 Concentrations in Serum Samples

ITIH5 concentrations in serum samples were determined using the ELISA kit for human ITIH5 commercialized by Uscn Life Science, Inc. (Houston, Tex.).

### Results

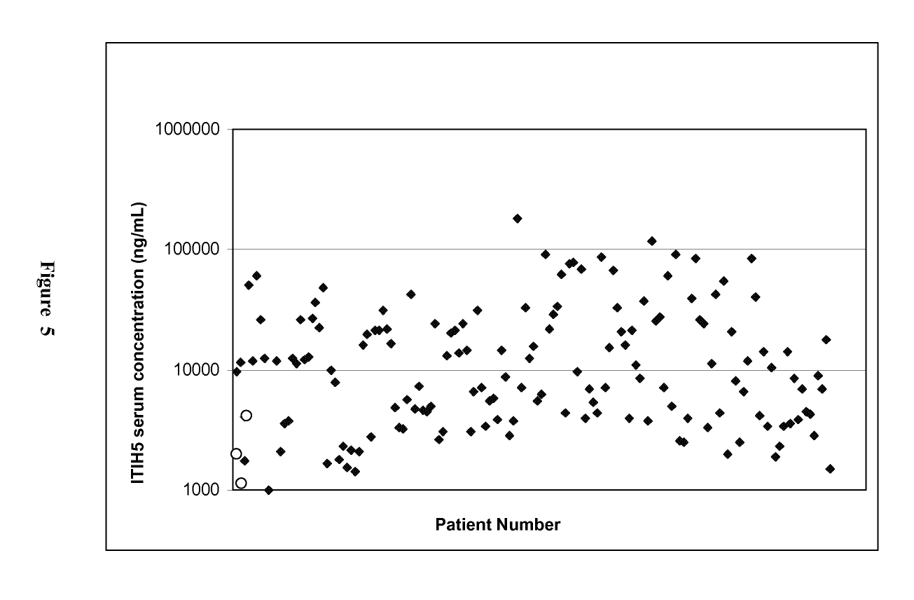

The results obtained are presented on FIG. 5. These results demonstrate that detection and quantification of ITIH5 in sera of human individuals is feasible, and that the diversity of physiopathological conditions of the female patients tested is reflected by the wide range of ITIH5 concentrations measured in the different samples. 34% of the individuals (including all the men (3)) tested exhibited an ITIH5 concentration of less than 5.000 ng/mL, 16.5% had an ITIH5 concentration between 5.000 and 10,000 ng/mL, 26% had an ITIH5 concentration between 10.000 to 25.000 ng/mL, 13.5% had an ITIH5 concentration between 25.000 and 50.000 ng/mL, 8.5% had an ITIH5 concentration between 50.000 and 100.000 ng/mL and 1.5% had an ITIH5 concentration of more than 100.000 ng/mL. These preliminary results show that the ITIH5 concentration in serum can vary from about 1000 ng/mL to about 180.000 ng/mL (i.e., by a factor of 1 to more than 100) depending on the physiopathological condition of the woman tested.

1. A method for detecting endometrial receptivity to embryo implantation in a female mammal, comprising steps of:
(a) measuring the level of expression of ITIH5 in a biological sample obtained from the female mammal; and
(b) correlating the level of expression of ITIH5 in the biological sample with endometrial receptivity to embryo implantation in said female subject.

2. A method for detecting the window of embryo implantation in a female mammal, comprising steps of:
(a) providing or obtaining a biological sample from said female mammal;
(b) measuring the expression level of ITIH5 in the biological sample obtained from the female mammal;
(c) repeating steps (a) and (b) with biological samples obtained from a plurality of stages of the menstrual or estrous cycle of said female mammal; and
(d) determining the period of time of the menstrual or estrous cycle when the endometrium of said female subject is available for embryo implantation, based on the levels of expression measured.

3. A method for diagnosing infertility in a female placental mammal, comprising steps of:
(a) measuring the level of expression of ITIH5 in a biological sample obtained from the female mammal; and
(b) correlating the level of expression of ITIH5 in the biological sample with infertility in said female mammal.

4. A method for monitoring the effects of a treatment protocol to induce ovarian hyperstimulation or ovulation induction on endometrium receptivity in a female mammal, comprising steps of:
(a) providing or obtaining a biological sample from said female mammal;
(b) measuring the expression level of ITIH5 in the biological sample obtained from the female mammal;
(c) repeating steps (a) and (b) with biological samples obtained from a plurality of stages of the menstrual or estrous cycle of said female mammal; and
(d) detecting and/or identifying, in said female mammal, an alteration of the endometrium receptivity due to the treatment, based on the expression level measured.

5. A method for assisted reproduction in a female placental mammal, comprising steps of:
(a) measuring the expression level of ITIH5 in a biological sample obtained from the female mammal;
(b) repeating step (a) with biological samples obtained from a plurality of stages of the menstrual or estrous cycle of said female mammal;
(c) correlating the level of expression of ITIH5 in one or more samples of step (b) with endometrial maturation; and
(d) transferring or introducing at least one embryo into the uterus of said female mammal when said endometrium is mature.

6. A method for assessing the probability of success of embryo implantation in the endometrium of a female mammal following a naturally achieved conception or a conception resulting from assisted reproduction technology, comprising steps of:
(a) measuring the level of expression of ITIH5 in a biological sample obtained from the female mammal; and
(b) correlating the level of expression of ITIH5 in said biological sample with the probability of success of embryo implantation in the endometrium of said female mammal.

7. A method for diagnosing pregnancy in female mammal, comprising steps of:
(a) measuring the level of expression of ITIH5 in a biological sample obtained from the female mammal; and
(b) correlating the level of expression of ITIH5 in the biological sample with embryo implantation and diagnosing pregnancy.

8. The method according to claim 7 further comprising a step of detecting and/or measuring the expression level of one or more hormones selected from the group consisting of hormone folliculostimulante (FSH), luteinizing hormone (LH), progesterone, anti-Müllerian hormone (AMH), estradiol, beta-human chorionic gonadotropin (β-hCG) and any combination thereof.

9. The method according to claim 1 further comprising detecting the level of expression of ITIH5 in biological samples obtained from a plurality of stages of the menstrual or estrous cycle of the female mammal.

10. The method according to claim 3 further comprising determining the period of time of the menstrual or estrous cycle when the endometrium of said female subject is available for embryo implantation, based on the levels of expression measured.

11. A method for the early diagnosis of endometriosis in a female mammal, comprising steps of:
(a) measuring the level of expression of ITIH5 in a biological sample obtained from the female mammal; and
(b) correlating the level of expression of ITIH5 in the biological sample with a diagnosis of endometriosis.

12. The method according to claim 1, wherein the female mammal is a woman of reproductive age or a domesticated placental mammal.

13. The method according to claim 1, wherein the biological sample is a sample of biological fluid selected from the group consisting of whole blood, serum, urine, uterine/cervical secretions, and vaginal secretions.

14. The method according to claim 13, wherein the biological sample is serum or urine.

15. The method according to claim 1, wherein measuring the level of expression of ITIH5 in a biological sample obtained from the female mammal comprises measuring the DNA or RNA expression of the ITIH5 gene.

16. The method according to claim 1, wherein measuring the level of expression of ITIH5 in a biological sample obtained from the female mammal comprises measuring the expression of a polypeptide encoded by the ITIH5 gene.

17. The method according to claim 16, wherein measuring the expression of a polypeptide encoded by the ITIH5 gene is performed using an ELISA or immunoassay.

18-21. (canceled)

22. A home pregnancy test kit comprising at least one test strip, wherein the at least one test strip is sensitive to the presence of ITIH5, in urine, and changes color, or otherwise indicates, when above the threshold sensitivity to ITIH5 is detected.
